(19) United States
(12) Patent Application Publication
(10) Pub. No.: US 2011/0144267 A1
(43) Pub. Date: Jun. 16, 2011

(54) FUNCTIONALIZED (METH)ACRYLATE MONOMER, POLYMER, COATING AGENT, AND PRODUCTION AND CROSS-LINKING METHOD

(75) Inventors: Thorben Schuetz, Seeheim-Jugenheim (DE); Joachim Knebel, Alsbach-Haehnlein (DE); Mario Gomez Andreu, Pfungstadt (DE); Christine Maria Breiner, Laudenbach (DE); Ralf Jackstell, Rostock (DE); Matthias Beller, Nienhagen (DE); Anne Grotevendt, Malchin (DE); Dirk Fridag, Haltern am See (DE); Robert Franke, Marl (DE)

(73) Assignee: Evonik Roehm GmbH, Darmstadt (DE)

(21) Appl. No.: 13/058,149

(22) PCT Filed: Sep. 4, 2009
(86) PCT No.: PCT/EP2009/061429

§ 371 Date: Feb. 8, 2011

(30) Foreign Application Priority Data
Sep. 8, 2008 (DE) 10 2008 046 076.1
Jun. 8, 2009 (DE) 10 2009 026 820.0

(51) Int. Cl. C08L 33/14; C08F 20/28; C08L 67/07; C08F 8/30; C08F 8/32; C07C 69/54

(52) U.S. Cl. 524/539; 526/315; 524/558; 525/376; 525/382; 560/205

(57) ABSTRACT

The present invention relates to a (meth)acrylate monomer of the general formula (I)

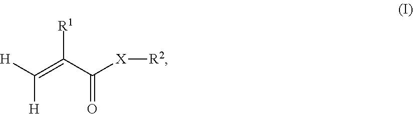

in which R¹ is hydrogen or a methyl group, X is oxygen or a group of the formula NR′ in which R′ is hydrogen or a radical having 1 to 6 carbon atoms, and R² is a radical having 3 to 31 carbon atoms and at least one aldehyde group.

The present invention further relates to a process for preparing the monomers set out above, to polymers obtainable from this monomer mixture, and to coating materials which comprise the stated polymers.

The present invention relates to a functionalized (meth)acrylate monomer and to a process for preparing it, and also to a monomer mixture which comprises a (meth)acrylate monomer. The present invention is further directed to a polymer obtainable using said monomer and/or said monomer mixture. The present invention further relates to a coating material and to a process for crosslinking.

Coating materials, more particularly paints and varnishes, have for a long time been produced synthetically. Relatively recent coating materials comprise polymers containing carbonyl groups, which can be cured by addition of crosslinking agents to give relatively solvent-resistant coatings. These coating materials are set out in publications including WO 94/025433. Nevertheless, improving the profile of properties of these coating materials is a continual requirement.

In view of the prior art it is now an object of the present invention to provide monomers which can be processed to polymers having outstanding properties. These properties include more particularly features which become apparent through coating materials and coatings which are obtainable from the coating materials.

More particularly the monomers ought to be able to be processed to dispersions and to polymers, emulsion polymers for example, which have a very low residual monomer content.

Additionally, therefore, it was an object of the present invention to provide a coating material which has a particularly long storage life and durability. The intention, furthermore, was that the hardness of the coatings obtainable from the coating materials should be able to be varied over a wide range. More particularly there was an intention that particularly hard, scratch-resistant coatings should be obtainable.

A further object is seen as being that of providing polymers which can be used to obtain coating materials without volatile organic solvents. The coatings obtainable from the coating materials ought to have high weathering stability, more particularly high UV resistance. Furthermore, the films obtainable from the coating materials ought to have a low tack after a short time.

Furthermore, the coatings obtainable from the polymers and monomer mixtures ought to be particularly highly resistant to solvents. This stability ought to be high with respect to a large number of different solvents. There ought likewise to be very good resistance to acidic and alkaline cleaning products.

Furthermore, therefore, it was an object of the present invention to specify monomers, polymers and coating materials which are obtainable in a particularly cost-effective way. With regard to the polymers it is noted that they ought to have a small fraction of monomers that are costly and inconvenient to prepare, without detriment to performance.

A further object can be seen as being that of providing a process for preparing these monomers that allows the product to be obtained very cost-effectively. Furthermore, the monomer obtained ought to contain only very small amounts of by-products and catalyst residues.

A further object of the invention was to provide a process with which the monomer can be obtained very selectively. The monomers obtainable in accordance with the present process ought to be able to be reacted without problems in further process steps without the need for costly and inconvenient purification.

It was an object of the present invention, furthermore, to provide processes for preparing monomers that can be carried out easily and cost-effectively. In these processes the product ought as far as possible to be obtained in high yields and, viewed comprehensively, with a low energy consumption.

These objects and others which, although not explicitly stated, are nevertheless readily inferrable or derivable from the circumstances discussed in the introduction are achieved by a monomer having all of the features of claim 1. Judicious modifications of the monomer of the invention are protected in dependent claims. With regard to a monomer mixture, to a polymer, to a coating material and to a process for preparing a monomer, claims 8, 15, 20 and 23 provide achievement of the underlying objects.

The present invention accordingly provides a (meth)acrylate monomer of the general formula (I)

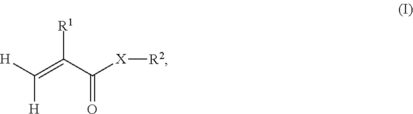

in which R¹ is hydrogen or a methyl group, X is oxygen or a group of the formula NR′ in which R′ is hydrogen or a radical having 1 to 6 carbon atoms, and R² is a radical having 3 to 31 carbon atoms and at least one aldehyde group.

Through the measures according to the invention it is additionally possible to obtain advantages including the following:

The monomer mixtures of the invention can be processed to polymers, coating materials and coatings which have a very low residual monomer content.

The hardness of the coatings obtainable from coating materials of the invention, which are based in turn on the polymers and/or monomer mixtures, can be varied over a wide range. In one preferred modification, in accordance with the invention, it is possible more particularly to obtain particularly hard, scratch-resistant coatings. The coatings obtainable from the coating materials of the present invention exhibit a surprisingly high solvent resistance, which is manifested more particularly in trials with methyl isobutyl ketone (MIBK) or ethanol. Thus the coatings obtained exhibit an outstanding classification in the context more particularly of trials in accordance with the DIN 68861-1 furniture test.

Coating materials obtainable using the monomer mixtures of the invention do not generally require any volatile organic solvents. Furthermore, the coating materials of the invention exhibit a high level of storage stability, a high durability and a very good storage life. In particular there is virtually no aggregate formed.

The coatings obtainable from the coating materials of the invention exhibit a high level of weathering stability, more particularly a high UV resistance. The films obtainable from the coating materials, furthermore, have a low tack after a short time.

The monomers, monomer mixtures, polymers and coating materials of the invention can be prepared inexpensively on a large scale. With regard to the polymers, it is noted that they may have a relatively low fraction of monomers that are costly and inconvenient to prepare, without detriment to performance. The performance of the polymers is apparent from, among other things, the properties of the coating materials and coatings obtainable therefrom.

The coating materials of the invention are eco-friendly and can be processed and produced safely and without great cost or inconvenience. The coating materials of the invention display a very high shearing stability.

The present invention further provides a process for preparing functionalized (meth)acrylates, in which the monomer is obtained very cost-effectively. Surprisingly the (meth)acrylate obtained contains only very small amounts of by-products, and in general there are no catalyst residues in the product mixture. Accordingly it is possible for a composition obtainable in accordance with the present process to be reacted in further process steps without problems, and without need for costly and inconvenient purification.

The process of the invention makes it possible, furthermore, to prepare functionalized (meth)acrylates in a way which is particularly selective.

Moreover, the process of the invention can be carried out simply and inexpensively, allowing the product to be obtained in high yields and, as viewed overall, with a low energy consumption.

The (meth)acrylate monomer of the invention is of the general formula (I)

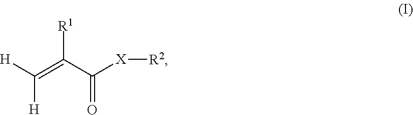

in which R¹ is hydrogen or a methyl group, X is oxygen or a group of the formula NR′ in which R′ is hydrogen or a radical having 1 to 6 carbon atoms, and R² is a radical having 3 to 31 carbon atoms and at least one aldehyde group.

The expression “radical having 1 to 6 carbon atoms” or “radical having 3 to 31 carbon atoms” stands respectively for a group which has 1 to 6 or 3 to 31 carbon atoms. It encompasses aromatic and heteroaromatic groups and also alkyl, cycloalkyl, alkoxy, cycloalkoxy, alkenyl, alkanoyl and alkoxycarbonyl groups, plus heteroaliphatic groups. The stated groups may be branched or unbranched. These groups may also have substituents, more particularly halogen atoms or hydroxyl groups.

The radicals R′ preferably stand for alkyl groups. The preferred alkyl groups are the methyl, ethyl, propyl, isopropyl, 1-butyl, 2-butyl, 2-methylpropyl and tert-butyl groups.

In formula (I) the radical R² is a group having 3 to 31 carbon atoms, more particularly having 3 to 25, preferably having 3 to 9, more preferably having 4 to 6 carbon atoms, which comprises at least one aldehyde group. According to a further embodiment of the present invention, (meth)acrylate monomers are preferred which have 10 to 25 carbon atoms. In this case the radical R² may comprise one, two, three or more aldehyde groups, it being possible for the radical R² to be substituted and for further functional groups to be present, examples being C—C double bonds. In one preferred modification of the present invention, the radical R² is an alkyl or alkenyl group which comprises one or two aldehyde groups, particular preference being given to radicals having one aldehyde group. This group may comprise heteroatoms, especially oxygen atoms and/or nitrogen atoms, in the form, for example, of an ester, ether, amino and/or amide group.

The preferred radicals R² include more particularly the 2-formylethyl, 3-formylethyl, 2-formylpropyl, 3-formylpropyl, 2-formylocta-7-enyl, 2,7-diformyloctyl, 9-formyloctadecyl and 10- formyloctadecyl group.

The preferred (meth)acrylate monomers of formula (I) include (meth)acrylate monomers having 3 to 9 carbon atoms in the radical R², such as, for example, 3-oxopropyl(meth)acrylate (2-formylethyl(meth)acrylate), 4-oxobutyl(meth)acrylate (3-formylpropyl(meth)acrylate), 2-methyl-3-oxopropyl(meth)acrylate (2-formyl-2-methylethyl(meth)acrylate), 2-formyloctenyl(meth)acrylate, 3-formyloctenyl(meth)acrylate, 8-formyloctenyl(meth)acrylate, 7-formyloctenyl(meth)acrylate, 2,8-diformyloctyl(meth)acrylate and 3,7-diformyloctyl(meth)acrylate.

The (meth)acrylate monomers of formula (I) further include (meth)acrylate monomers having 10 to 25 carbon atoms in the radical R², such as (meth)acrylates which derive from fatty acids, fatty alchols and fatty acid amides, such as 9-formyloctadecan-12-enyl(meth)acrylate, 9,12-diformyloctadecyl(meth)acrylate, 12-formyloctadecan-6,9-dienyl(meth)acrylate, 9-formylhexadecyl(meth)acrylate, 10-formylhexadecyl(meth)acrylate, 9-formyloctadecyl(meth)acrylate, 10-formyloctadecyl(meth)acrylate, (meth)acryloyloxy-2-hydroxypropyl-9-formyloctadecanoic ester, (meth)acryloyloxy-2-hydroxypropyl-10-formyloctadecanoic ester, (meth)acryloyloxy-2-hydroxypropyl-9-formyloctadecanamide and/or (meth)acryloyloxy-2-hydroxypropyl-10-formyloctadecanamide.

The (meth)acrylate monomers of formula (I) further include (meth)acrylates which are of the formula (II)

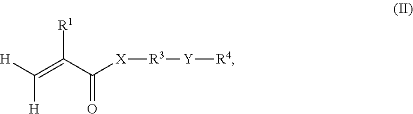

in which R¹ is hydrogen or a methyl group, X is oxygen or a group of the formula NR′ in which R′ is hydrogen or a radical having 1 to 6 carbon atoms, R³ is an alkylene group having 1 to 22 carbon atoms, Y is oxygen, sulphur or a group of the formula NR″ in which R″ is hydrogen or a radical having 1 to 6 carbon atoms, and R⁴ is a radical having 8 carbon atoms and at least one aldehyde group.

Preferably the radicals R′ and R″ are alkyl groups. The preferred alkyl groups include the methyl, ethyl, propyl, isopropyl, 1-butyl, 2-butyl, 2-methylpropyl and tert-butyl group.

In formula (II) the radical R³ is an alkylene group having 1 to 22 carbon atoms, preferably having 1 to 10, more preferably having 2 to 6 carbon atoms. In one particular embodiment of the present invention the radical R³ is an alkylene group having 2 to 4, more preferably 2, carbon atoms. The alkylene groups having 1 to 22 carbon atoms include more particularly the methylene, ethylene, propylene, isopropylene, n-butylene, isobutylene, tert-butylene or cyclohexylene group, the ethylene group being particularly preferred.

The radical R⁴ comprises at least one aldehyde group, preferably two aldehyde groups. In a further aspect the radical R⁴ is a group having an aldehyde group and a double bond.

The (meth)acrylates of formula (II) include, for example, 2-[(2-formylocta-7-enyl)methylamino]ethyl 2-methylprop-2-enoate; 2-[(7-formylocta-2-enyl)methylamino]ethyl 2-methylprop-2-enoate; 2-[(3-formylocta-7-enyl)methylamino]ethyl 2-methylprop-2-enoate; 2-[(8-formylocta-2-enyl)methylamino]ethyl 2-methylprop-2-enoate; 2-[(2,7-diformyloctyl)methylamino]ethyl 2-methylprop-2-enoate; 2-[(3,7-diformyloctyl)methylamino]ethyl 2-methylprop-2-enoate; 2-[(2,8-diformyloctyl)methylamino]ethyl 2-methylprop-2-enoate; 2-[(3,8-diformyloctyl)methylamino]ethyl 2-methylprop-2-enoate; 2-[(2-formylocta-7-enyl)methylamino]ethyl(meth)acrylamide; 2-[(7-formylocta-2-enyl)methylamino]ethyl(meth)acrylamide; 2-[(3-formylocta-7-enyl)methylamino]ethyl(meth)acrylamide; 2-[(8-formylocta-2-enyl)methylamino]ethyl(meth)acrylamide; 2-[(2,7-diformyloctypmethylamino]ethyl(meth)acrylamide; 2-[(3,7-diformyloctyl)methylamino]ethyl(meth)acrylamide; 2-[(2,8-diformyloctyl)methylamino]ethyl(meth)acrylamide; 2-[(3,8-diformyloctyl)methylamino]ethyl(meth)acrylamide; (meth)acryloyloxy-2-hydroxypropyl-9-formyloctadeca-12-enoic ester; (meth)acryloyloxy-2-hydroxypropyl-12-formyloctadeca-9-enoic ester; (meth)acryloyloxy-2-hydroxypropyl-10-formyloctadeca-12-enoic ester; (meth)acryloyloxy-2-hydroxypropyl-13-formyloctadeca-9-enoic ester; (meth)acryloyloxy-2-hydroxypropyl-9,12-diformyloctadecanoic ester; (meth)acryloyloxy-2-hydroxypropyl-10,13-diformyloctadecanoic ester; (meth)acryloyloxy-2-hydroxypropyl-9-formyloctadecanoic ester; (meth)acryloyloxy-2-hydroxypropyl-10-formyloctadecanoic ester; (meth)acryloyloxy-2-hydroxypropyl-9-formyloctadeca-12-enamide; (meth)acryloyloxy-2-hydroxypropyl-12-formyloctadeca-9-enamide; (meth)acryloyloxy-2-hydroxypropyl-10-formyloctadeca-12-enamide (meth)acryloyloxy-2-hydroxypropyl-13-formyloctadeca-9-enamide; (meth)acryloyloxy-2-hydroxypropyl-9,12-diformyloctadecanamide; (meth)acryloyloxy-2-hydroxypropyl-10,13-diformyloctadecanamide; (meth)acryloyloxy-2-hydroxypropyl-9-formyloctadecanamide; and/or (meth)acryloyloxy-2-hydroxypropyl-10-formyloctadecanamide.

The stated monomers may be used individually or as a mixture of two or more compounds.

(Meth)acrylate monomers of formula (I) can be obtained, with advantages which a person skilled in the art could not have foreseen, by reacting a reactant of the formula (III)

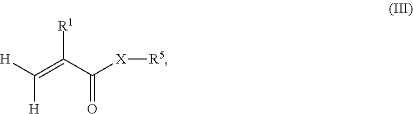

in which X is oxygen or a group of the formula NR′ in which R′ is hydrogen or a radical having 1 to 6 carbon atoms and R⁵ is an unsaturated radical having at least one double bond and 2 to 30 carbon atoms, preferably 2 to 24 carbon atoms, with carbon monoxide and hydrogen in the presence of a catalyst.

Reactions of unsaturated compounds with carbon monoxide and hydrogen in the presence of a catalyst are often referred to as hydroformylation processes. The preferred catalysts include more particularly compounds which comprise rhodium, iridium, palladium and/or cobalt, with rhodium being particularly preferred.

In one particular embodiment it is possible more particularly to use complexes which comprise at least one phosphorus compound as ligand for the catalysis. Preferred phosphorus compounds comprise aromatic groups and at least one but more preferably two phosphorus atoms. The phosphorus compounds include more particularly phosphines, phosphites, phosphinites, phosphonites. Examples of phosphines are triphenylphosphine, tris(p-tolyl)phosphine, tris(m-tolyl)phosphine, tris(o-tolyl)phosphine, tris(p-methoxyphenyl)phosphine, tris(p-dimethylaminophenyl)phosphine, tricyclohexylphosphine, tricyclopentylphosphine, triethylphosphine, tri-(1-naphthyl)phosphine, tribenzylphosphine, tri-n-butylphosphine, tri-tert-butylphosphine. Examples of phosphites are trimethyl phosphite, triethyl phosphite, tri-n-propyl phosphite, triisopropyl phosphite, tri-n-butyl phosphite, triisobutyl phosphite, tri-tert-butyl phosphite, tris(2-ethylhexyl) phosphite, triphenyl phosphite, tris(2,4-di-tert-butylphenyl)phosphite, tris(2-tert-butyl-4-methoxyphenyl)phosphite, tris(2-tert-butyl-4-methylphenyl)phosphite, tris(p-cresyl) phosphite. Examples of phosphonites are methyldiethoxyphosphine, phenyldimethoxyphosphine, phenyldiphenoxyphosphine, 2-phenoxy-2H-dibenz[c,e][1,2]oxaphosphorine and its derivatives in which some or all of the hydrogen atoms have been replaced by alkyl and/or aryl radicals or halogen atoms. Common phosphinite ligands are diphenyl(phenoxy)phosphine and its derivatives diphenyl(methoxy)phosphine and diphenyl(ethoxy)phosphine.

The particularly preferred ligands include more particularly 4,5-bis(diphenylphosphino)-9,9-dimethylxanthene (Xantphos) and its derivative 10,10′-(2,7-di-tert-butyl-9,9-dimethyl-9H-xanthene-4,5-diyl)bis(10H-phenoxaphosphinine) (POP-Xantphos) and Biphephos

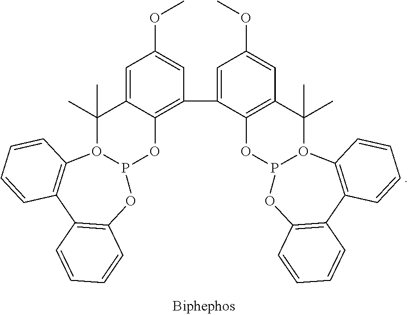

Catalysts and ligands for the hydroformylation are set out for example in WO 2008/071508 A1, filed on Nov. 13, 2007 at the European Patent Office with the application number PCT/EP2007/062248; EP 982 314 B1, filed on Aug. 17, 1999 at the European Patent Office with the application number 99116208; WO 2008/012128 A1, filed on May 29, 2007 at the European Patent Office with the application number PCT/EP2007/055165; WO 2008/006633 A1, filed on May 11, 2007 at the European Patent Office with the application number PCT/EP2007/054576; WO 2007/036424 A1, filed on Sep. 8, 2006 at the European Patent Office with the application number PCT/EP2006/066181; WO 2007/028660 A1, filed on Hyn. 2, 2006 at the European Patent Office with the application number PCT/EP2006/062872; WO 2005/090276 A1, filed on Jan. 27, 2005 at the European Patent Office with the application number PCT/EP2005/050347, reference being made to these publications for disclosure purposes, and the catalysts and ligands disclosed therein being incorporated into the present specification.

In one particular embodiment of the present process, the phosphorus compound employed as ligand may be used in excess over the metal. Through this embodiment it is possible to achieve surprising advantages in respect of selectivity and reactivity. Preferably the ratio of metal to ligand can be in the range from 1:1 to 1:1000, more preferably in the range from 1:2 to 1:200.

The preferred starting materials which can be used for preparing the (meth)acrylates of formula (I) and which conform to formula (II) above include (meth)acrylates having 2 to 8 carbon atoms in the alkyl radical, and deriving from unsaturated alcohols, and (meth)acrylates having 9 to 24 carbon atoms in the alkyl radical and containing at least one double bond.

The (meth)acrylates having 2 to 8 carbon atoms in the alkyl radical and deriving from unsaturated alcohols include 2-propynyl(meth)acrylate, allyl(meth)acrylate and vinyl(meth)acrylate.

The (meth)acrylates having 9 to 30 carbon atoms, preferably 9 to 24 carbon atoms, in the alkyl radical and at least one double bond in the alkyl radical include more particularly (meth)acrylates which derive from unsaturated fatty acids, fatty alcohols and fatty acid amides, such as heptadecenyloyloxy-2-ethyl(meth)acrylamide, heptadecan-dien-yloyloxy-2-ethyl(meth)acrylamide, heptadecan-trien-yloyloxy-2-ethyl(meth)acrylamide, heptadecenyloyloxy-2-ethyl(meth)acrylamide, (meth)acryloyloxy-2-ethylpalmitoleamide, (meth)acryloyloxy-2-ethyloleamide, (meth)acryloyloxy-2-ethylicosenamide, (meth)acryloyloxy-2-ethylcetoleamide, (meth)acryloyloxy-2-ethylerucamide, (meth)acryloyloxy-2-ethyllinoleamide, (meth)acryloyloxy-2-ethyllinoleneamide, (meth)acryloyloxy-2-propylpalmitoleamide, (meth)acryloyloxy-2-propyloleamide, (meth)acryloyloxy-2-propylicosenamide, (meth)acryloyloxy-2-propylcetoleamide, (meth)acryloyloxy-2-propylerucamide, (meth)acryloyloxy-2-propyllinoleamide and (meth)acryloyloxy-2-propyllinoleneamide; (meth)acryloyloxy-2-hydroxypropyllinoleic ester, (meth)acryloyloxy-2-hydroxypropyllinolenic ester and (meth)acryloyloxy-2-hydroxypropyloleic ester, octadecan-dien-yl(meth)acrylate, octadecan-trien-yl(meth)acrylate, hexadecenyl(meth)acrylate, octadecenyl(meth)acrylate and hexadecan-dien-yl(meth)acrylate.

The (meth)acrylates having 9 to 30 carbon atoms, preferably 9 to 24 carbon atoms, in the alkyl radical and at least one double bond in the alkyl radical further include, more particularly, (meth)acrylate monomers of the general formula (IV)

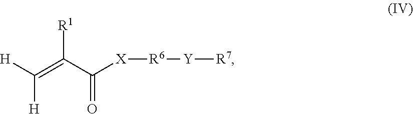

in which R¹ is hydrogen or a methyl group, X is oxygen or a group of the formula NR′ in which R′ is hydrogen or a radical having 1 to 6 carabon atoms, R⁶ is an alkylene group having 1 to 22 carbon atoms, Y is oxygen, sulphur or a group of the formula NR″ in which R″ is hydrogen or a radical having 1 to 6 carbon atoms, and R⁷ is an unsaturated radical having 8 carbon atoms and at least two double bonds. For preferred embodiments of the radical R⁶, refer to the remarks relating to the radical R³, since these radicals correspond to one another.

The (meth)acrylate monomers of the general formula (IV) include 2-[((2-E)octa-2,7-dienyl)methylamino]ethyl 2-methylprop-2-enoate, 2-[((2-Z)octa-2,7-dienyl)methylamino]ethyl 2-methylprop-2-enoate, 2-[((3-E)octa-3,7-dienyl)methylamino]ethyl 2-methylprop-2-enoate, 2-[((4-Z)octa-4,7-dienyl)methylamino]ethyl 2-methylprop-2-enoate, 2-[(octa-2,6-dienyl)methylamino]ethyl 2-methylprop-2-enoate, 2-[(octa-2,4-dienyl)methylamino]ethyl 2-methylprop-2-enoate, 2-[(octa-3,5-dienyl)methylamino]ethyl 2-methylprop-2-enoate, 2-[((2-E)octa-2,7-dienyl)methylamino]ethyl(meth)acrylamide, 2-[((2-Z)octa-2,7-dienyl)methylamino]ethyl(meth)acrylamide, 2-[((3-E)octa-3,7-dienyl)methylamino]ethyl(meth)acrylamide, 2-[((4-Z)octa-4,7-dienyl)methylamino]ethyl(meth)acrylamide, 2-[(octa-2,6-dienyl)methylamino]ethyl(meth)acrylamide, 2-[(octa-2,4-dienyl)methylamino]ethyl(meth)acrylamide, 2-[(octa-3,5-dienyl)methylamino]ethyl(meth)acrylamide, 2-[((2-E)octa-2,7-dienyl)ethylamino]ethyl 2-methylprop-2-enoate, 2-[((2-Z)octa-2,7-dienyl)ethylamino]ethyl 2-methylprop-2-enoate, 2-[((3-E)octa-3,7-dienyl)ethylamino]ethyl 2-methylprop-2-enoate, 2-[((4-Z)octa-4,7-dienyl)ethylamino]ethyl 2-methylprop-2-enoate, 2-[(octa-2,6-dienyl)ethylamino]ethyl 2-methylprop-2-enoate, 2-[(octa-2,4-dienyl)ethylamino]ethyl 2-methylprop-2-enoate, 2-[(octa-3,5-dienyl)ethylamino]ethyl 2-methylprop-2-enoate, 2-[((2-E)octa-2,7-dienyl)methylamino]ethyl prop-2-enoate, 2-[((2-Z)Octa-2,7-dienyl)methylamino]ethyl prop-2-enoate, 2-[((3-E)octa-3,7-dienyl)methylamino]ethyl prop-2-enoate, 2-[((4-Z)octa-4,7-dienyl)methylamino]ethyl prop-2-enoate, 2-[(octa-2,6-dienyl)methylamino]ethyl prop-2-enoate, 2-[(octa-2,4-dienyl)methylamino]ethyl prop-2-enoate, 2-[(octa-3,5-dienyl)methylamino]ethyl prop-2-enoate, 2-((2-E)octa-2,7-dienyloxy)ethyl 2-methylprop-2-enoate, 2-((2-Z)octa-2,7-dienyloxy)ethyl 2-methylprop-2-enoate, 2-((3-E)octa-3,7-dienyloxy)ethyl 2-methylprop-2-enoate, 2-((4-Z)octa-4,7-dienyloxy)ethyl 2-methylprop-2-enoate, 2-(octa-2,6-dienyloxy)ethyl 2-methylprop-2-enoate, 2-(octa-2,4-dienyloxy)ethyl 2-methylprop-2-enoate, 2-(octa-3,5-dienyloxy)ethyl 2-methylprop-2-enoate, 2-((2-E)octa-2,7-dienyloxy)ethyl prop-2-enoate, 2-((2-Z)octa-2,7-dienyloxy)ethyl prop-2-enoate, 2-((3-E)octa-3,7-dienyloxy)ethyl prop-2-enoate, 2-((4-Z)octa-4,7-dienyloxy)ethyl prop-2-enoate, 2-(octa-2,6-dienyloxy)ethyl prop-2-enoate, 2-(octa-2,4-dienyloxy)ethyl prop-2-enoate and 2-(octa-3,5-dienyloxy)ethyl prop-2-enoate.

The reactants of formula (Ill) may be used individually or as a mixture.

The (meth)acrylates set out above that can be used as reactants are in some cases available commercially. Furthermore, these (meth)acrylates can be obtained by telomerization reactions, by reaction of fatty acids with glycidyl(meth)acrylate, by esterification reactions or transesterification reactions.

Reactions of glycidyl(meth)acrylate with fatty acids are set out in publications including WO 2006/01361.

The (meth)acrylates set out above can be obtained more particularly by processes in which methacrylic acid, acrylic acid or a mixture thereof, also referred to below for short as (meth)acrylic acid, or a (meth)acrylate, more particularly methyl(meth)acrylate or ethyl(meth)acrylate, is reacted with an alcohol and/or with an amine. Transesterifications of alcohols with (meth)acrylates or the preparation of (meth)acrylamides are set out, moreover, in CN 1355161, DE 21 29 425, filed on Jun. 14, 1971 at the German Patent Office with the application number P 2129425.7, DE 34 23 443 filed on 26.06.84 at the German Patent Office with the application number P 3423443.8, EP-A-0 534 666 filed on Sep. 16, 1992 at the European Patent Office with the application number EP 92308426.3, or DE 34 30 446 filed on Aug. 18, 1984 at the German Patent Office with the application number P 3430446.0, the reaction conditions described in these publications and also the catalysts etc. set out therein being incorporated for purposes of disclosure into the present specification. Furthermore, these reactions are described in “Synthesis of Acrylic Esters by Transesterification”, J. Haken, 1967.

The reactant to be reacted with the (meth)acrylic acid or the (meth)acrylate may zo advantageously be of the formula (V)

H—X—R⁶—Y—R⁷ (V),

in which X is oxygen or a group of the formula NR′ in which R′ is hydrogen or a radical having 1 to 6 carbon atoms, R⁶ is an alkylene group having 1 to 22 carbon atoms, Y is oxygen, sulphur or a group of the formula NR″ in which R″ is hydrogen or a radical having 1 to 6 carbon atoms, and R⁷ is an at least doubly unsaturated radical having 8 carbon atoms.

With regard to the definition of preferred radicals R′, R″, R⁶, Y and R⁷, reference is made to the description of the formula (IV).

The preferred reactants of formula (V) include (methyl(octa-2,7-dienyl)amino)ethanol, (ethyl(octa-2,7-dienyl)amino)ethanol, 2-octa-2,7-dienyloxyethanol, (methyl(octa-2,7-dienyl)amino)ethylamine, (methyl(octa-3,7-dienyl)amino)ethanol, (ethyl(octa-3,7-dienyl)amino)ethanol, 2-octa-3,7-dienyloxyethanol, (methyl(octa-3,7-dienyl)amino)ethylamine, (methyl(octa-4,7-dienyl)amino)ethanol, (ethyl(octa-4,7-dienyl)amino)ethanol, 2-octa-4,7-dienyloxyethanol, (methyl(octa-4,7-dienyl)amino)ethylamine, (methyl(octa-5,7-dienyl)amino)ethanol, (ethyl(octa-5,7-dienyl)amino)ethanol, 2-octa-5,7-dienyloxyethanol, (methyl(octa-5,7-dienyl)amino)ethylamine, (methyl(octa-2,6-dienyl)amino)ethanol, (ethyl(octa-2,6-dienyl)amino)ethanol, 2-octa-2,6-dienyloxyethanol, (methyl(octa-2,6-dienyl)amino)ethylamine, (methyl(octa-2,5-dienyl)amino)ethanol, (ethyl(octa-2,5-dienyl)amino)ethanol, 2-octa-2,5-dienyloxyethanol, (methyl(octa-2,5-dienyl)amino)ethylamine, (methyl(octa-2,4-dienyl)amino)ethanol, (ethyl(octa-2,4-dienyl)amino)ethanol, 2-octa-2,4-dienyloxyethanol, (methyl(octa-2,4-dienyl)amino)ethylamine, (methyl(octa-3,6-dienyl)amino)ethanol, (ethyl(octa-3,6-dienyl)amino)ethanol, 2-octa-3,6-dienyloxyethanol, (methyl(octa-3,6-dienyl)amino)ethylamine, (methyl(octa-3,5-dienyl)amino)ethanol, (ethyl(octa-3,5-dienyl)amino)ethanol, 2-octa-3,5-dienyloxyethanol, (methyl(octa-3,5-dienyl)amino)ethylamine, (methyl(octa-4,6-dienyl)amino)ethanol, (ethyl(octa-4,6-dienyl)amino)ethanol, 2-octa-4,6-dienyloxyethanol and (methyl(octa-4,6-dienyl)amino)ethylamine. The reactants of formula (II) can be used individually or as a mixture.

The reactants of formula (V) can be obtained by methods including known methods of the telomerization of 1,3-butadiene. The term “telomerization” here denotes the reaction of compounds having conjugated double bonds in the presence of nucleophiles. The processes set out in publications WO 2004/002931, filed on Jul. 17, 2003 at the European Patent Office with the application number PCT/EP2003/006356, WO 03/031379, filed on Oct. 1, 2002 with the application number PCT/EP2002/10971, and WO 02/100803, filed on May 4, 2002 with the application number PCT/EP2002/04909, more particularly the catalysts used for the reaction and the reaction conditions, such as pressure and temperature, for example, are incorporated for purposes of disclosure into the present specification.

The telomerization of 1,3-butadiene may take place preferably using metal compounds which comprise metals of groups 8 to 10 of the periodic table of the elements as catalysts, it being possible with particular preference to use palladium compounds, more particularly palladium-carbene complexes, which are set out in greater detail in the publications listed above.

The nucleophiles used may more particularly be dialcohols, such as ethylene glycol, 1,2-propanediol and 1,3-propanediol; diamines, such as ethylenediamine, N-methylethylenediamine, N,N′-dimethylethylenediamine or hexamethylenediamine; or amino alcanols, such as aminoethanol, n-methylaminoethanol, N-ethylaminoethanol, aminopropanol, N-methylaminopropanol or N-ethylaminopropanol.

When using (meth)acrylic acid as a nucleophile it is possible for example to obtain octadienyl(meth)acrylates which are suitable as a reactant for preparing the monomer of formula (I).

The temperature at which the telomerization reaction is performed is between 10 and 180° C., preferably between 30 and 120° C., more preferably between 40 and 100° C. The reaction pressure is 1 to 300 bar, preferably 1 to 120 bar, more preferably 1 to 64 bar and very preferably 1 to 20 bar.

The preparation of isomers of compounds which have an octa-2,7-dienyl group can be accomplished by isomerizing the double bonds which are present in the compounds with an octa-2,7-dienyl group.

Besides the reactant or reactants of formula (III) and the catalysts described above, the reaction is carried out using hydrogen (H₂) and carbon monoxide (CO). Preferably the reaction can be carried out at an overall gas pressure in the range from 1 to 200 bar, more preferably in the range from 1 to 150 bar, with particular preference in the range from 1 to 100 bar.

In accordance with one particular aspect of the present invention, the hydrogen pressure at which reaction is carried out may be greater than the pressure of the carbon monoxide.

The temperature at which the reaction of the reactant of formula (III) with hydrogen and carbon monoxide is carried out is not critical per se. Particular advantages can be achieved more particularly by carrying out the reaction at a temperature in the range from 20 to 250° C., preferably from 40 to 200° C., more preferably in the range from 150 to 160° C.

In accordance with one particular aspect of the present invention, the reaction can be carried out in an inert organic solvent. These solvents include, for example, aromatic hydrocarbons, such as toluene or xylene, dioxane, carboxylic esters, such as ethyl acetate, for example. With particular advantage the reaction can be carried out substantially without the use of an inert organic solvent. In that case it is more particularly the reactants and also the ligands that form the medium in which the reaction is performed.

Surprising advantages can additionally be obtained through the use of a stabilizer. The preferred stabilizers include hydroquinones, hydroquinone ethers, such as hydroquinone monomethyl ether, or di-tert-butylpyrocatechol, phenothiazine, methylene blue or sterically hindered phenols, an example being 2,4-dimethyl-6-tert-butylphenol, as are widely known in the art. These compounds can be used individually or in the form of mixtures, and in general are available commercially. For further details refer to the relevant technical literature, more particularly to Römpp-Lexikon Chemie; editors: J. Falbe, M. Regitz; Stuttgart, New York; 10th edition (1996); entry heading “Antioxidantien” and the references cited in that entry.

The monomer set out above of the formula (I) can be used with advantage in a monomer mixture which comprises one or more monomers which are copolymerizable with the monomer of formula (I).

Advantages which are not obvious per se to a person skilled in the art can be achieved through a monomer mixture which comprises at least 0.5% by weight, preferably at least 2% by weight and more preferably at least 5% by weight of monomers of the formula (I), based on the total weight of the monomer mixture.

Besides at least one (meth)acrylate monomer of formula (I), the monomer mixture comprises at least one further monomer which is copolymerizable. These copolymerizable monomers include monomers having an acid group, monomers A comprising ester groups and different from the monomers of the formula I, and styrene monomers.

Monomers containing acid groups are compounds which can be copolymerized preferably free-radically with the (meth)acrylate monomers of formula (I) set out above. They include, for example, monomers having a sulphonic acid group, such as vinylsulphonic acid, for example; monomers having a phosphonic acid group, such as vinylphosphonic acid, for example; and unsaturated carboxylic acids, such as methacrylic acid, acrylic acid, fumaric acid and maleic acid, for example. Methacrylic acid and acrylic acid are particularly preferred. The monomers containing acid groups can be used individually or as a mixture of two, three or more monomers containing acid groups.

The preferred monomers A comprising ester groups include, in particular, (meth)acrylates which differ from the monomers of formula (I), and also fumarates, maleates and/or vinyl acetate. The expression (meth)acrylates encompasses methacrylates and acrylates and also mixtures of both. These monomers are widely known.

These include, more particularly, (meth)acrylates having 1 to 6 carbon atoms in the alkyl radical and deriving from saturated alcohols, such as methyl(meth)acrylate, ethyl(meth)acrylate, n-propyl(meth)acrylate, isopropyl(meth)acrylate, n-butyl(meth)acrylate, tert-butyl(meth)acrylate and pentyl(meth)acrylate, hexyl(meth)acrylate and cycloalkyl(meth)acrylates, such as cyclopentyl(meth)acrylate, cyclohexyl(meth)acrylate.

Particular preference is given to using mixtures, to prepare polymers comprising methacrylates and acrylates. Thus it is possible more particularly to use mixtures of methyl methacrylate and acrylates having 2 to 6 carbons, such as ethyl acrylate, butyl acrylate and hexyl acrylate.

These comonomers further include, for example, (meth)acrylates having at least 7 carbon atoms in the alkyl radical and deriving from saturated alcohols, such as, for example, 2-ethylhexyl(meth)acrylate, heptyl(meth)acrylate, 2-tert-butylheptyl(meth)acrylate, octyl(meth)acrylate, 3-isopropylheptyl(meth)acrylate, nonyl(meth)acrylate, decyl(meth)acrylate, undecyl(meth)acrylate, 5-methylundecyl(meth)acrylate, dodecyl(meth)acrylate, 2-methyldodecyl(meth)acrylate, tridecyl(meth)acrylate, 5-methyltridecyl(meth)acrylate, tetradecyl(meth)acrylate, pentadecyl(meth)acrylate, hexadecyl(meth)acrylate, 2-methyihexadecyl(meth)acrylate, heptadecyl(meth)acrylate, 5-isopropylheptadecyl(meth)acrylate, 4-tert-butyloctadecyl(meth)acrylate, 5-ethyloctadecyl(meth)acrylate, 3-isopropyloctadecyl(meth)acrylate, octadecyl(meth)acrylate, nonadecyl(meth)acrylate, eicosyl(meth)acrylate, cetyleicosyl(meth)acrylate, stearyleicosyl(meth)acrylate, docosyl(meth)acrylate and/or eicosyltetratriacontyl(meth)acrylate; cycloalkyl(meth)acrylates, such as 3-vinylcyclohexyl(meth)acrylate, bornyl(meth)acrylate, cycloalkyl(meth)acrylates, such as 2,4,5-tri-t-butyl-3-vinylcyclohexyl(meth)acrylate, 2,3,4,5-tetra-t-butylcyclohexyl(meth)acrylate; heterocyclic(meth)acrylates, such as 2-(1-imidazolyl)ethyl(meth)acrylate, 2-(4-morpholinyl)ethyl(meth)acrylate and 1-(2-methacryloyloxyethyl)-2-pyrrolidone; nitriles of (meth)acrylic acid and other nitrogen-containing methacrylates, such as N-(methacryloyloxyethyl)diisobutylketimine, N-(methacryloyloxyethyl)dihexadecylketimine, methacryloylamidoacetonitrile, 2-methacryloyloxyethylmethylcyanamide, cyanomethyl methacrylate; aryl(meth)acrylates, such as benzyl(meth)acrylate or phenyl(meth)acrylate, it being possible for each of the aryl radicals to be unsubstituted or to be substituted up to four times; (meth)acrylates which contain two or more (meth)acrylic groups, glycol di(meth)acrylates, such as ethylene glycol di(meth)acrylate, diethylene glycol di(meth)acrylate, triethylene glycol di(meth)acrylate, tetra- and polyethylene glycol di(meth)acrylate, 1,3-butanediol(meth)acrylate, 1,4-butanediol(meth)acrylate, 1,6-hexanediol di(meth)acrylate, glycerol di(meth)acrylate; dimethacrylates of ethoxylated bisphenol A; (meth)acrylates having three or more double bonds, such as glycerol tri(meth)acrylate, trimethylolpropane tri(meth)acrylate, pentaerythritol tetra(meth)acrylate and dipentaerythritol penta(meth)acrylate, and (meth)acrylates deriving from saturated fatty acid amides, such as pentadecyloyloxy-2-ethyl(meth)acrylamide, heptadecyloyloxy-2-ethyl(meth)acrylamide, (meth)acryloyloxy-2-ethyllauramide, (meth)acryloyloxy-2-ethylmyristamide, (meth)acryloyloxy-2-ethylpalmitamide, (meth)acryloyloxy-2-ethylstearamide, (meth)acryloyloxy-2-propyllauramide, (meth)acryloyloxy-2-propylmyristamide, (meth)acryloyloxy-2-propylpalmitamide and (meth)acryloyloxy-2-propylstearamide.

Furthermore, the monomers A comprising ester groups include (meth)acrylates of formula (III) which can be used more particularly as starting compounds for preparing the present (meth)acrylates of formula (I).

The monomers A comprising ester groups further include vinyl esters, such as vinyl acetate;

maleic acid derivatives, such as, for example, maleic anhydride, esters of maleic acid, for example dimethyl maleate, methylmaleic anhydride; and fumaric acid derivatives, such as dimethyl fumarate.

A further preferred group of comonomers are styrene monomers, such as, for example, styrene, substituted styrenes having an alkyl substituent in the side chain, such as, for example, α-methylstyrene and α-ethylstyrene, substituted styrenes having an alkyl substituent on the ring, such as vinyltoluene and p-methylstyrene, and halogenated styrenes, such as monochlorostyrenes, dichlorostyrenes, tribromostyrenes and tetrabromostyrenes, for example.

Besides the monomers set out above it is possible for polymers of the invention obtained by the polymerization of monomer mixtures to contain further monomers. These include, for example, heterocyclic vinyl compounds, such as 2-vinylpyridine, 3-vinylpyridine, 2-methyl-5-vinylpyridine, 3-ethyl-4-vinylpyridine, 2,3-dimethyl-5-vinylpyridine, vinylpyrimidine, vinylpiperidine, 9-vinylcarbazole, 3-vinylcarbazole, 4-vinylcarbazole, 1-vinylimidazole, 2-methyl-1-vinylimidazole, N-vinylpyrrolidone, 2-vinylpyrrolidone, N-vinylpyrrolidine, 3-vinylpyrrolidine, N-vinylcaprolactam, N-vinylbutyrolactam, vinyloxolane, vinylfuran, vinylthiophene, vinylthiolane, vinylthiazoles and hydrogenated vinylthiazoles, vinyloxazoles and hydrogenated vinyloxazoles;

maleimide, methylmaleimide;

vinyl ethers and isoprenyl ethers; and

vinyl halides, such as vinyl chloride, vinyl fluoride, vinylidene chloride and vinylidene fluoride, for example.

Preferred monomer mixtures of the present invention comprise

0.1% to 90%, preferably 0.5% to 30%, more preferably 1%-10%, in particular 1%-6%, by weight of (meth)acrylate monomer of formula (I);

10% to 99.9%, preferably 40% to 90%, by weight of monomers with ester groups A;

0% to 20%, preferably 1% to 8%, more particulary 1% to 3%, by weight of monomer having an acid group,

0% to 70%, preferably 0% to 50%, more particularly 0% to 30%, by weight of styrene monomers, and

0% to 50%, preferably 0% to 30%, by weight of further comonomers, the amounts being based in each case on the total weight of the monomers.

In accordance with one particular aspect of the present invention it is possible in particular to use mixtures of monomers with ester groups A, these mixtures comprising monomers with ester groups that derive from saturated alcohols, and monomers of formula (III). The fraction of monomers of formula (III) is preferably in the range from 0.1% to 50%, more preferably in the range from 0.2% to 20% and very preferably in the range from 1% to 10%, by weight, based on the total weight of the monomers.

Particular preference is given to using mixtures which have a high fraction of monomers with ester groups A that derive from saturated alcohols having 1 to 6 carbon atoms. The fraction of monomers with ester groups A that derive from saturated alcohols having 1 to 6 carbon atoms is preferably in the range from 10% to 99.9%, more preferably in the range from 40% to 90% and very preferably in the range from 50% to 80%, by weight, based on the total weight of the monomers.

The (meth)acrylate monomers of formula (I) and monomer mixtures of the invention serve in particular for preparing or for modifying polymers. The polymerization can take place in any known way. Such ways include, in particular, free-radical, cationic or anionic polymerization, in which context it is also possible to employ variants of these polymerization processes, such as, for example, ATRP (=Atom Transfer Radical Polymerization), NMP processes (Nitroxide Mediated Polymerization) or RAFT (=Reversible Addition Fragmentation chain Transfer).

The polymers obtainable by these means are new and therefore likewise provided by the present invention. The polymers of the invention comprise at least one unit derived from a (meth)acrylate monomer of the general formula (I). As already described, the monomers of the invention can be reacted by free-radical polymerization. Therefore the term “unit” arises from the reaction of a double bond, with two covalent bonds being constructed. Typically these units are also referred to as repeating units, if there are two or more of these units present in a polymer.

The aforementioned monomers or monomer mixtures can be reacted, for example, by solution polymerizations, bulk polymerizations or emulsion polymerizations, it being possible to achieve surprising advantages by means of a free-radical emulsion polymerization.

Methods of emulsion polymerization are set out in sources including Ullmann's Encyclopedia of Industrial Chemistry, Fifth Edition. The general approach for this is to prepare an aqueous phase which as well as water may include typical additives, more particularly emulsifiers and protective colloids for stabilizing the emulsion.

This aqueous phase is then admixed with monomers, and polymerization is carried out in the aqueous phase. When preparing homogeneous polymer particles, it is possible here to add a monomer mixture batchwise or continuously over a time interval.

The emulsion polymerization can be implemented for example as a miniemulsion or as a microemulsion, and these are set out in more detail in Chemistry and Technology of Emulsion Polymerisation, A. M. van Herk (editor), Blackwell Publishing, Oxford 2005 and J. O'Donnell, E. W. Kaler, Macromolecular Rapid Communications 2007, 28(14), 1445-1454. A miniemulsion is usually characterized by the use of costabilizers or swelling agents, and often long-chain alkanes or alkanols are used. The droplet size in the case of miniemulsions is preferably in the range from 0.05 to 20 μm. The droplet size in the case of microemulsions is situated preferably in the range below 1 μm, allowing particles to be obtained with a size below 50 nm. In the case of microemulsions use is often made of additional surfactants, examples being hexanol or similar compounds.

The dispersing of the monomer-containing phase in the aqueous phase can take place using known agents. These include, more particularly, mechanical methods and also the application of ultrasound.

In the preparation of homogeneous emulsion polymers it is possible with preference to use a monomer mixture which comprises 1% to 50%, more preferably 1% to 10%, especially 1%-6%, by weight of (meth)acrylate monomer of formula (I).

When preparing core-shell polymers it is possible to change the composition of the monomer mixture in steps, polymerization preferably taking place, before the composition is changed, to a conversion of at least 80% by weight, more preferably at least 95% by weight, based in each case on the total weight of the monomer mixture used. A core-shell polymer here is a polymer which has been prepared by a two-stage or multi-stage emulsion polymerization, without the core-shell construction having been shown by means, for example, of electron microscopy. The progress of the polymerization reaction in each step can be monitored in a known way, such as by gravimetry or gas chromatography, for example.

The monomer composition for preparing the core comprises preferably 50% to 100% by weight of (meth)acrylates, particular preference being given to the use of a mixture of acrylates and methacrylates. After the core has been prepared, it is possible to graft or to polymerize onto the core, preferably, a monomer mixture which comprises 1% to 50%, more preferably 1% to 20%, especially 2%-10%, by weight of (meth)acrylate monomer of formula (I).

The emulsion polymerization is conducted preferably at a temperature in the range from 0 to 120° C., more preferably in the range from 30 to 100° C. Polymerization temperatures which have proved to be especially favourable in this context are temperatures in the range from greater than 60 to less than 90° C., judiciously in the range from greater than 70 to less than 85° C., preferably in the range from greater than 75 to less than 85° C.

The polymerization is initiated with the initiators that are customary for emulsion polymerization. Suitable organic initiators are, for example, hydroperoxides such as tert-butyl hydroperoxide or cumene hydroperoxide. Suitable inorganic initiators are hydrogen peroxide and also the alkali metal salts and the ammonium salts of peroxodisulphuric acid, more particularly ammonium, sodium and potassium peroxodisulphate. Suitable redox initiator systems are, for example, combinations of tertiary amines with peroxides or sodium disulphite and alkali metal salts and the ammonium salts of peroxodisulphuric acid, more particularly sodium and potassium peroxodisulphate. Further details can be taken from the technical literature, more particularly H. Rauch-Puntigam, Th. Völker, “Acryl- and Methacrylverbindungen”, Springer, Heidelberg, 1967 or Kirk-Othmer, Encyclopedia of Chemical Technology, Vol. 1, pages 386ff., J. Wiley, New York, 1978. Particular preference in the context of the present invention is given to the use of organic and/or inorganic initiators.

The stated initiators may be used both individually and in a mixture. They are preferably used in an amount of 0.05% to 3.0% by weight, based on the total weight of the monomers of the respective stage. It is also possible with preference to carry out the polymerization with a mixture of different polymerization initiators having different half-lives, in order to keep the flow of free radicals constant over the course of the polymerization and also at different polymerization temperatures.

Stabilization of the batch is accomplished preferably by means of emulsifiers and/or protective colloids. The emulsion is preferably stabilized by emulsifiers, in order to obtain a low dispersion viscosity. The total amount of emulsifier is preferably 0.1% to 15% by weight, more particularly 1% to 10% by weight and more preferably 2% to 5% by weight, based on the total weight of the monomers used. In accordance with one particular aspect of the present invention it is possible to add a portion of the emulsifiers during the polymerization.

Particularly suitable emulsifiers are anionic or nonionic emulsifiers or mixtures thereof, more particularly
alkyl sulphates, preferably those having 8 to 18 carbon atoms in the alkyl radical, alkyl and alkylaryl ether sulphates having 8 to 18 carbon atoms in the alkyl radical and 1 to 50 ethylene oxide units;
sulphonates, preferably alkylsulphonates having 8 to 18 carbon atoms in the alkyl radical, alkylarylsulphonates having 8 to 18 carbon atoms in the alkyl radical, esters and monoesters of sulphosuccinic acid with monohydric alcohols or alkylphenols having 4 to 15 carbon atoms in the alkyl radical; where appropriate these alcohols or alkylphenols may also have been ethoxylated with 1 to 40 ethylene oxide units;
phosphoric acid partial esters and their alkali metal and ammonium salts, preferably alkyl and alkylaryl phosphates having 8 to 20 carbon atoms in the alkyl or alkylaryl radical and 1 to 5 ethylene oxide units;
alkyl polyglycol ethers, preferably having 8 to 20 carbon atoms in the alkyl radical and 8 to 40 ethylene oxide units;
alkylaryl polyglycol ethers, preferably having 8 to 20 carbon atoms in the alkyl or alkylaryl radical and 8 to 40 ethylene oxide units;
ethylene oxide/propylene oxide copolymers, preferably block copolymers, favourably having 8 to 40 ethylene and/or propylene oxide units.

The particularly preferred anionic emulsifiers include, more particularly, fatty alcohol ether sulphates, diisooctyl sulphosuccinate, lauryl sulphate, C15-paraffinsulphonate, it being possible to use these compounds generally in the form of the alkali metal salt, more particularly the sodium salt. These compounds may be obtained commercially, more particularly, under the commercial designations Disponil® FES 32, Aerosol® OT 75, Texapon® K1296 and Statexan® K1 from the companies Cognis GmbH, Cytec Industries, Inc. and Bayer AG.

Judicious nonionic emulsifiers include tert-octyiphenol ethoxylate with 30 ethylene oxide units and fatty alcohol polyethylene glycol ethers which have preferably 8 to 20 carbon atoms in the alkyl radical and 8 to 40 ethylene oxide units. These emulsifiers are available commercially under the commercial designations Triton® X 305 (Fluka), Tergitol® 15-S-7 (Sigma-Aldrich Co.), Marlipal® 1618/25 (Sasol Germany) and Marlipal® O13/400 (Sasol Germany).

With preference it is possible to use mixtures of anionic emulsifier and nonionic emulsifier. The weight ratio of anionic emulsifier to nonionic emulsifier can judiciously be in the range from 20:1 to 1:20, preferably 2:1 to 1:10 and more preferably 1:1 to 1:5.

Mixtures which have proven to be especially appropriate are those comprising a sulphate, more particularly a fatty alcohol ether sulphate, a lauryl sulphate, or a sulphonate, more particularly a diisooctyl sulphosuccinate or a paraffin-sulphonate, as anionic emulsifier, and an alkylphenol ethoxylate or a fatty alcohol polyethylene glycol ether having in each case preferably 8 to 20 carbon atoms in the alkyl radical and 8 to 40 ethylene oxide units as nonionic emulsifier.

Where appropriate the emulsifiers can also be used in a mixture with protective colloids. Suitable protective colloids include partially hydrolysed polyvinyl acetates, polyvinylpyrrolidones, carboxymethyl, methyl, hydroxyethyl and hydroxypropyl cellulose, starches, proteins, poly(meth)acrylic acid, poly(meth)acrylamide, polyvinylsulphonic acids, melamine-formaldehyde sulphonates, naphthalene-formaldehyde sulphonates, styrene-maleic acid and vinyl ether-maleic acid copolymers. If protective colloids are used they are used preferably in an amount of 0.01% to 1.0% by weight, based on the total amount of the monomers. The protective colloids may be included in the initial charge before the start of the polymerization, or metered in. The initiator may be included in the initial charge or metered in. It is also possible, furthermore, to include a portion of the initiator in the initial charge and to meter in the remainder.

The polymerization is preferably started by heating the batch to the polymerization temperature and including the initiator in the initial charge and/or adding it as a metered feed, preferably in aqueous solution. Some of the monomers may be included in the initial charge to the reactor, and the remainder metered in over a defined period of time. Generally it is advantageous to polymerize the portion of the monomers that has been included in the initial charge to the reactor, and only then to begin the feed. As an alternative to including a defined amount of monomer in the initial charge, the feed may be interrupted for a number of minutes after, for example 1%-5% of the monomers have been metered in. The metered feeds of emulsifier and monomers may be implemented separately or, preferably, as a mixture, more particularly as an emulsion in water.

The emulsion polymerization may be carried out in a broad pH range. The pH is preferably between 2 and 9. In one particular embodiment the polymerization is carried out at pH values between 4 and 8, more particularly between 6 and 8. It is also possible to adjust the dispersion after polymerization to a pH range which is preferred for the application. For pigmented coating systems, the range is generally 8-9 or above.

Within wide limits, the molecular weight of the polymers is to start with not critical. Where particularly hard and solvent-resistant coating materials having good mechanical properties are desired, then a very high molecular weight may be useful. Preferred emulsion polymers with a high fraction of polymers which are insoluble in THF may be obtained in the manner set out above. The reaction parameters for obtaining a high molecular weight are known. Thus in this case it is possible in particular to do without the use of molecular weight regulators.

Paints and varnishes which have particularly good and simple processing qualities may also contain polymers with a relatively low molecular weight, the solvent resistance and the hardness of these coatings attaining a relatively high level. These polymers with a particularly good processing quality may preferably have a molecular weight below 1 000 000 g/mol, preferably below 500 000 g/mol and more preferably below 250 000 g/mol. The molecular weight can be determined by means of gel permeation chromatography (GPC) against a PMMA standard.

Polymers, especially emulsion polymers, with a low molecular weight can be obtained by the addition of molecular weight regulators to the reaction mixture before or during the polymerization. For this purpose it is possible to use sulphur-free molecular weight regulators and/or sulphur-containing molecular weight regulators.

The sulphur-free molecular weight regulators include, for example - without wishing to impose any restriction—dimeric α-methylstyrene (2,4-diphenyl-4-methyl-1-pentene), enol ethers of aliphatic and/or cycloaliphatic aldehydes, terpenes, β-terpinene, terpinolene, 1,4-cyclohexadiene, 1,4-dihydronaphthalene, 1,4,5,8-tetrahydronaphthalene, 2,5-dihydrofuran, 2,5-dimethylfuran and/or 3,6-dihydro-2H-pyran, preference being given to dimeric α-methylstyrene.

As sulphur-containing molecular weight regulators it is possible with preference to use mercapto compounds, dialkyl sulphides, dialkyl disulphides and/or diaryl sulphides. The following polymerization regulators are named by way of example: di-n-butyl sulphide, di-n-octyl sulphide, diphenyl sulphide, thiodiglycol, ethylthioethanol, diisopropyl disulphide, di-n-butyl disulphide, di-n-hexyl disulphide, diacetyl disulphide, diethanol sulphide, di-tert-butyl trisulphide and dimethyl sulphoxide. Compounds used preferably as molecular weight regulators are mercapto compounds, dialkyl sulphides, dialkyl disulphides and/or diaryl sulphides. Examples of these compounds are ethyl thioglycolate, 2-ethylhexyl thioglycolate, cysteine, 2-mercaptoethanol, 1,3-mercaptopropanol, 3-mercaptopropane-1,2-diol, 1,4-mercaptobutanol, mercaptoacetic acid, 3-mercaptopropionic acid, mercaptosuccinic acid, thioglycerol, thioacetic acid, thiourea and alkyl mercaptans such as n-butyl mercaptan, n-hexyl mercaptan or n-dodecyl mercaptan. Polymerization regulators used with particular preference are mercapto alcohols and mercapto carboxylic acids.

The molecular weight regulators are used preferably in amounts of 0.05% to 10%, more preferably 0.1% to 5%, by weight, based on the monomers used in the polymerization. In the polymerization it is of course also possible to employ mixtures of polymerization regulators.

Furthermore it is possible to employ polymerizations using molecular weight regulators for the purpose of reducing the minimum film formation temperature (MFFT) of the polymers obtainable by the polymerization. In accordance with this preferred embodiment the fraction of molecular weight regulators can be calculated such that the polymers and/or the coating materials of the invention have a minimum film formation temperature (MFFT) of not more than 60° C., more preferably not more than 50° C. and very preferably not more than 40° C., which can be measured in accordance with DIN ISO 2115. The higher the fraction of molecular weight regulator, the lower the minimum film formation temperature.

One of the ways in which the adjustment of the particle radii can be influenced is via the fraction of emulsifiers. The higher this fraction, more particularly at the beginning of the polymerization, the smaller the particles obtained.

The polymers obtainable in accordance with the process described above, especially the emulsion polymers obtainable with preference, represent further subject matter of the present invention.

Preferably the emulsion polymer is non-crosslinked or is crosslinked to such a small extent that the fraction of the weight of the emulsion polymer that is soluble in tetrahydrofuran (THF) at 20° C. is more than 60% by weight. In a further, preferred embodiment, the emulsion polymer can have a fraction of 2% to 60%, more preferably 10% to 50% and very preferably 20% to 40%, by weight, based on the weight of the emulsion polymer, which is soluble in THF at 20° C. To determine the soluble fraction, a sample of the polymer that has been dried in the absence of oxygen is stored in 200 times the amount of solvent, based on the weight of the sample, at 20° C. for 4 h. In order to ensure the absence of oxygen, the sample, for example, can be dried under nitrogen or under reduced pressure. Subsequently the solution is separated, by filtration for example, from the insoluble fraction. After the solvent has been evaporated the weight of the residue is determined. For example, a 0.5 g sample of an emulsion polymer dried under reduced pressure can be stored in 150 ml of THF for 4 hours.

In accordance with one preferred modification of the present invention an emulsion polymer may exhibit swelling of at least 800%, more preferably at least 1200% and very preferably at least 1300% in tetrahydrofuran (THF) at 20° C. The upper limit on the swelling is not critical per se, the swelling preferably being not more than 5000%, more preferably not more than 3000% and very preferably not more than 2500%. To determine the swelling, a sample of the emulsion polymer that has been dried in the absence of oxygen is stored in 200 times the amount of THF at 20° C. for 4 hours. As a result the sample swells. The swollen sample is separated from the supernatant solvent. Subsequently the solvent is removed from the sample. For example, a major fraction of the solvent can be evaporated at room temperature (20° C.). Solvent residues can be removed in a drying oven (140° C.), generally over the course of 1 hour. From the weight of the solvent absorbed by the sample and the weight of the dry sample the swelling is obtained. Furthermore, the difference in the weight of the sample prior to the swelling experiment and the weight of the dried sample after the swelling experiment produces the soluble fraction of the emulsion polymer.

The particle radius of the emulsion polymers can be within a wide range. Thus, in particular, it is possible to use emulsion polymers having a particle radius in the range from 10 to 500 nm, preferably 10 to 100 nm, particularly preferably 20 to 60 nm. More particularly, particle radii of less than 50 nm may be advantageous for film formation and coating properties. The radius of the particles can be determined by means of PCS (Photon Correlation Spectroscopy), the data given relating to the d50 value (50% of the particles are smaller, 50% are larger). This can be done using, for example, a Beckman Coulter N5 Submicron Particle Size Analyzer.

The glass transition temperature of the polymer of the invention is situated preferably in the range from −30° C. to 70° C., more preferably in the range from −20 to 40° C. and very preferably in the range from 0 to 25° C. The glass transition temperature may be influenced via the nature and the fraction of the monomers used to prepare the polymer. The glass transition temperature, Tg, of the polymer may be determined in a known way by means of Differential Scanning calorimetry (DSC). Moreover, the glass transition temperature Tg may also be calculated approximately in advance by means of the Fox equation. According to Fox T. G., Bull. Am. Physics Soc. 1, 3, page 123 (1956) it is the case that:

$$\frac{1}{Tg} = \frac{{x}_{1}}{{Tg}_{1}} + \frac{{x}_{2}}{{Tg}_{2}} + … + \frac{{x}_{n}}{{Tg}_{n}}$$

where xₙ represents the mass fraction (% by weight/100) of the monomer n and Tgₙ identifies the glass transition temperature, in kelvin, of the homopolymer of the monomer n. Further useful information can be found by the skilled person in the Polymer Handbook, 2nd Edition, J. Wiley & Sons, New York (1975), which gives Tg values for the most common homopolymers. The polymer here may have one or more different glass transition temperatures. These figures therefore apply to a segment obtainable by polymerizing at least one (meth)acrylate monomer of formula (I), preferably a monomer mixture of the invention.

For many applications and properties the architecture of the polymer is not critical. The polymers, especially the emulsion polymers, may accordingly comprise random copolymers, gradient copolymers, block copolymers and/or graft copolymers. Block copolymers and gradient copolymers can be obtained, for example, by discontinuously altering the monomer composition during chain propagation. In accordance with one preferred aspect of the present invention the emulsion polymer comprises a random copolymer in which the monomer composition over the polymerization is substantially constant. Since, however, the monomers may have different copolymerization parameters, the precise composition may fluctuate over the polymer chain of the polymer.

The polymer may constitute a homogeneous polymer which, for example, in an aqueous dispersion forms particles having a consistent composition. In this case the polymer, which is preferably an emulsion polymer, may be composed of one or more segments obtainable by polymerizing at least one (meth)acrylate monomer of formula (I), preferably a monomer mixture of the invention.

In accordance with another embodiment the emulsion polymer may constitute a core-shell polymer, which may have one, two, three or more shells. In this case the segment obtainable by polymerizing the monomer mixture of the invention or the (meth)acrylate monomer of formula (I) preferably forms the outermost shell of the core-shell polymer. The shell may be connected to the core or to the inner shells via covalent bonds. Moreover, the shell may also be polymerized onto the core or onto an inner shell. In this embodiment the segment obtainable by polymerizing the monomer mixture of the invention may in many cases be separated and isolated from the core by means of suitable solvents.

The weight ratio of segment obtainable by polymerizing the monomer mixture of the invention or the (meth)acrylate monomer of formula (I) to core may be situated preferably in the range from 6:1 to 1:6. Where the glass transition temperature of the core is higher than that of the shell, a ratio of 6:1 to 2:1 is particularly preferred; in the opposite case, 1:1 to 1:5 is particularly preferred.

The core may be formed preferably of polymers comprising 50% to 100%, preferably 60% to 90%, by weight of units derived from (meth)acrylates. Preference here is given to esters of (meth)acrylic acid whose alcohol residue comprises preferably 1 to 30 carbon atoms, more preferably 1 to 20 carbon atoms and very preferably 1 to 10 carbon atoms. They include, more particularly, (meth)acrylates deriving from saturated alcohols, such as methyl(meth)acrylate, ethyl(meth)acrylate, n-propyl(meth)acrylate, isopropyl(meth)acrylate, n-butyl(meth)acrylate, tert-butyl(meth)acrylate, pentyl(meth)acrylate and hexyl(meth)acrylate.

In accordance with one particular embodiment of the present invention the core can be prepared using a mixture which comprises methacrylates and acrylates. Thus it is possible more particularly to use mixtures of methyl methacrylate and acrylates having 2 to 6 carbons, such as ethyl acrylate, butyl acrylate and hexyl acrylate.

Furthermore, the polymers of the core may comprise the comonomers set out above. In accordance with one preferred modification the core may be crosslinked. This crosslinking may be achieved through the use of monomers having two, three or more free-radically polymerizable double bonds.

The shell of an emulsion polymer of the present invention that is obtainable by polymerizing a monomer mixture of the invention may comprise preferably 2% to 50% by weight of units derived from (meth)acrylate monomers of formula (I).

In accordance with one particular aspect the core may preferably have a glass transition temperature in the range from −30 to 200° C., more particularly in the range from −20 to 150° C. Particular preference is given to a glass transition temperature >50° C., more particularly >100° C. The shell of the emulsion polymer of the invention, preferably obtainable by polymerizing the monomer mixture of the invention, may preferably have a glass transition temperature in the range from −30° C. to 70° C., more preferably in the range from −20 to 40° C. and very preferably in the range from 0 to 25° C. In accordance with one particular aspect of the present invention the glass transition temperature of the core may be greater than the glass transition temperature of the shell. Judiciously the glass transition temperature of the core may be at least 10° C., preferably at least 20° C., above the glass transition temperature of the shell.

Surprising advantages are obtainable if the iodine number of the polymers of the invention is preferably in the range from 0.1 to 300 g iodine per 100 g polymer, more preferably in the range from 1 to 270 g iodine per 100 g polymer and very preferably 5 to 250 g iodine per 100 g polymer, measured in accordance with DIN 53241-1. The iodine number may also be measured more particularly on the basis of a dispersion of the invention.

Judiciously the polymer may have an acid number in the range from 0 to 50 mg KOH/g, preferably 0.1 to 40 mg KOH/g, more preferably 1 to 20 mg KOH/g and very preferably in the range from 2 to 10 mg KOH/g. The acid number may be determined in accordance with DIN EN ISO 2114 also from a dispersion.

The hydroxyl number of the polymer can be situated preferably in the range from 0 to 200 mg KOH/g, more preferably 1 to 100 mg KOH/g and very preferably in the range from 3 to 50 mg KOH/g. The hydroxyl number may be determined in accordance with DIN EN ISO 4629 also from a dispersion.

The polymers obtainable by polymerizing (meth)acrylate monomers of formula (I) or a monomer mixture of the invention can be isolated. In accordance with one particular embodiment of the present invention, the dispersions obtainable by emulsion polymerization can be employed as they are, as coating materials.

Coating materials which comprise the above polymers or compounds obtainable by reactions with the above (meth)acrylate monomers are likewise provided by the present invention. Coating materials are compositions which are suitable for the coating of substrates. The coating materials of the invention are crosslinkable by means of crosslinking agents. Moreover, preferred coating materials exhibit a tendency to self-crosslinking. Crosslinked films often feature a high solvent resistance. Particularly preferred coating materials are oxidatively crosslinkable, and so crosslinked films are produced from the coating materials on exposure to oxygen. The oxidatively crosslinkable coating materials preferably comprise polymers with unsaturated side chains, which are obtainable more particularly through use of monomers of formula (III) when preparing the polymers, or through use of monomers of formula (I) which additionally contain C—C double bonds in the radical R².

Besides the coating materials which comprise above polymers, it is also possible with success to use coating materials which are based on alkyd resins which have been modified with the (meth)acrylate monomers of the invention or the monomer mixtures of the invention. The term “modification” here is to be understood broadly, and so encompasses alkyd resins which contain one or more units, or repeating units, derived from the (meth)acrylate monomers of formula (I). Also embraced by the concept of “modification” are alkyd resins or alkyd resin dispersions which comprise the polymers set out above.

Alkyd resins are well established, the term referring generally to resins obtained by condensing polybasic carboxylic acids and polyhydric alcohols, these compounds generally being modified with long-chain alcohols (fatty alcohols), fatty acids or compounds containing fatty acid, examples being fats or oils (DIN 55945; 1968). Alkyd resins are described for example in Ullmann's Encyclopedia of Industrial Chemistry, 5th edition on CD-ROM. Besides these conventional alkyd resins it is also possible to use resins which have similar properties. These resins are likewise distinguished by a high level of groups derived from long-chain alcohols (fatty alcohols), fatty acids and/or compounds containing fatty acid, examples being fats or oils. These derivatives, however, do not necessarily contain polybasic carboxylic acids, but may instead be obtained, for example, by reaction of polyols with isocyanates. The alkyd resins that can be employed may be diluted or mixed preferably with water.

Preferred polybasic carboxylic acids for preparing the alkyd resins to be used with preference in the dispersion of the invention include dicarboxylic and tricarboxylic acids, such as, for example, phthalic acid, isophthalic acid, 5-(sodiumsulpho)isophthalic acid, terephthalic acid, trimellitic acid, 1,4-cyclohexanedicarboxylic acid, butanedioic acid, maleic acid, fumaric acid, sebacic acid, adipic acid and azelaic acid. These acids can also be used as anhydrides for the preparation. Particular preference is given to using aromatic dicarboxylic acids to prepare the alkyd resins. The fraction of polybasic carboxylic acids is preferably in the range from 2% to 50% by weight, more preferably 5% to 40% by weight, based on the weight of the reactants for preparing the resin that are used in the reaction mixture.

The alkyd resins are additionally prepared using polyhydric alcohols. These alcohols include trimethylolpropane, pentaerythritol, dipentaerythritol, trimethylolethane, neopentylglycol, ethylene glycol, 1,3-butanediol, 1,4-butanediol, 1,6-hexanediol, 1,4-cyclohexyldimethanol, diethylene glycol, triethylene glycol, polyethylene glycol, polytetrahydrofuran, polycaprolactonediol, polycaprolactonetriol, trimethylol monoallyl ether, trimethylol diallyl ether, pentaerythritol triallyl ether, pentaerythritol diallyl ether, pentaerythritol monoallyl ether, 2-ethyl-2-(hydroxymethyl)-1,3-propanediol, 2-methyl-1,3-propanediol, 2,2,4-trimethylpentanediol, 2,2,4-trimethyl-1,3-pentanediol, 2,2′-bis(4-hydroxycyclohexyl)propane(hydrogenated bisphenol A), propylene glycol, dipropylene glycol, polypropylene glycol, glycerol, and sorbitol. Particular preference among these is given to trimethylolethane, trimethylolpropane, pentaerythritol and sorbitol. In one particular aspect preference is given more particularly to alchols having three or more hydroxyl groups. The fraction of polyhydric alcohols is preferably in the range from 2% to 50% by weight, more preferably 5% to 40% by weight, based on the weight of the reactants for preparing the resin that are used in the reaction mixture.

Furthermore it is possible in particular to use fatty acids to prepare the alkyd resins set out above. In this case use may be made more particularly of saturated and unsaturated fatty acids, particular preference being given to mixtures which comprise unsaturated fatty acids. Preferred fatty acids have 6 to 30, more preferably 10 to 26 and very preferably 12 to 22 carbon atoms. The fraction of fatty acids is preferably in the range from 2% to 90% by weight, more preferably 10% to 70% by weight, based on the weight of the reactants for preparing the resin that are used in the reaction mixture.

The suitable saturated fatty acids include caprylic acid, capric acid, lauric acid, myristic acid, palmitic acid, margaric acid, arachidic acid, behenic acid, lignoceric acid, cerotinic acid, palmitoleic acid and stearic acid.

The preferred unsaturated fatty acids include undecylenoic acid, palmitoleic acid, oleic acid, elaidic acid, vaccenic acid, icosenoic acid, cetoleic acid, erucic acid, nervonic acid, linoleic acid, linolenic acid, arachidonic acid, timnodonic acid, clupanodonic acid and/or cervonic acid.

The fatty acids set out above may also be used, furthermore, in the form of their esters, as for example in the form of triglycerides.

The alkyd resins set out above may, furthermore, contain additional components. Examples of such components include monobasic carboxylic acids, monohydric alcohols or compounds which lead to emulsifying groups in the resins, such as polyethylene oxides, for example. The alkyd resins may further contain hydroxy carboxylic acids, such as 2-, 3- and 4-hydroxybenzoic acid, rizinoleic acid, dihydroxypropionic acid, dihydroxysuccinic acid, dihydroxybenzoic acid, 2,2-dimethylolacetic acid, 2,2-dimethylolpropionic acid, 2,2-dimethylolbutteric acid and 2,2-dimethylolpentanoic acid.

Additionally it is also possible to use modified alkyd resins which have been modified with resins, especially rosin, with styrene polymers, with acrylic polymers, with epoxides, with urethanes, with polyamides and/or with silicones. These modifications are set out in references which include the above-recited patent literature and Ullmann's Encyclopedia of Industrial Chemistry 5th edition on CD-ROM. Through these embodiments it is possible to modify more particularly the initial drying, the adhesive strength, the weathering stability, the storage life, the chemical resistance, the volume curing, the sag resistance of the wet film, and the abrasion resistance.

By way of example it is possible with preference to use alkyd resins which have been modified with polymers obtainable by free-radical polymerization. Resins of this kind are disclosed in publications including U.S. Pat. No. 5,538,760, U.S. Pat. No. 6,369,135 and DE-A-199 57 161. The resins set out in publication U.S. Pat. No. 5,538,760, filed on 22.05.95 at the Patent Office of the United States of America (USPTO) with the application No. 446,130, are included for purposes of disclosure in the present application. The resins set out in publication U.S. Pat. No. 6,369,135 B1, filed on Aug. 13, 1996 at the Patent Office of the United States of America (USPTO) with the application Ser. No. 08/696,361, are included for purposes of disclosure in the present application. The resins set out in publication DE-A-199 57 161, filed on Nov. 27, 1999 at the German Patent and Trademark Office (DPMA) with the application number DE 19957161.9, are included for purposes of disclosure in the present specification.

According to publications U.S. Pat. No. 5,538,760 and U.S. Pat. No. 6,369,135, modified alkyd resins can be obtained by methods including the polymerization of a monomer mixture in the presence of an alkyd resin. The weight ratio of monomer mixture to alkyd resin in this case is preferably in the range from 100:1 to 1:4, preferably 5:1 to 1:1.

Particularly judicious are resins including the acrylate-modified alkyd resins described in DE-A-199 57 161. These alkyd resins, in addition to an alkyd core, contain groups obtained by polymerizing (meth)acrylates.

These acrylate-modified alkyd resins can be prepared by - operating in the presence of at least one water-miscible diol

(1) dispersing in water at least one alkyd resin which, based on its total amount, contains 0.1% to 10% by weight of pendant and/or terminal allyloxy groups, to give dispersion 1,

(2) subjecting a mixture of methacrylic acid and at least one further, carboxyl-free olefinically unsaturated monomer to graft copolymerization in dispersion 1, to give dispersion 2, and

(3) once or n times, subjecting

(3.1) at least one acid-group-free olefinically unsaturated monomer and/or

(3.2) at least one mixture of at least one acid-group-containing olefinically unsaturated monomer and at least one acid-group-free olefinically unsaturated monomer to graft copolymerization in the dispersion 2 or 2 to n-1 resulting from the respective previous process step (2) or (2) to (n-1), with the proviso that, in step (3) of the process or in its repetitions (3) to (n), acid groups are incorporated in an amount corresponding in total to not more than 90 mol % of the amount of acid groups incorporated in step (2) of the process.

The aforementioned pendant and/or terminal allyloxy group may be present in the alkyd resin in an amount, based in each case on the alkyd resin, of 0.1% to 10%, preferably 0.2% to 9%, more preferably 0.3% to 8%, very preferably 0.4% to 7%, with very particular preference 0.5% to 6% and in particular 0.6% to 5% by weight. The oxygen atom of the allyloxy group may be part of a urethane group, an ester group or an ether group that connects the allyl radical to the main chain of the alkyd resin.

Examples of suitable compounds for introducing pendant and/or terminal allyloxy groups are allyl alcohol, 2-hydroxyethyl allyl ether, 3-hydroxypropyl allylether, trimethylolpropane monoallyl or diallyl ether, glycerol monoallyl or diallyl ether, pentaerythritol monoallyl, diallyl or triallyl ether, mannitol monoallyl, diallyl, triallyl or tetraallyl ether, allyl esters of dihydroxypropionic, dihydroxysuccinic, dihydroxybenzoic, 2,2-dimethylolacetic, 2,2-dimethylolpropionic, 2,2-dimethylolbutyric or 2,2-dimethylolpentanoic acids, or allylurethane, among which advantage is possessed by trimethylolpropane monoallyl ether. For the modification with acrylates, dispersion 1 can be subjected in a step (2) to graft copolymerization with methacrylic acid and at least one further olefinically unsaturated monomer. The further olefinically unsaturated monomers may, in addition to the olefinically unsaturated double bonds, also contain reactive functional groups, with the exception of carboxyl groups - for example isocyanate-reactive, carbamate-reactive, N-methylol- or N-methylol ether reactive or alkoxycarbonylamino reactive groups. It is essential here that these reactive functional groups, under the prevailing reaction conditions and during the subsequent storage of the dispersions of the invention, do not enter into any reactions with the carboxyl groups of the methacrylic acid or with other reactive functional groups that may be present. One example of reactive functional groups which meet these requirements is the hydroxyl group. These monomers are known per se, with examples being set out in DE 199 57 161. They include more particularly hydroxyalkyl esters of acrylic acid, of methacrylic acid or of another alpha, beta-olefinically unsaturated carboxylic acid, esters of acrylic acid, of methacrylic acid, of crotonic acid or of ethacrylic acid with up to 20 carbon atoms in the alkyl radical.

Preference extends to alkyd resins obtainable in accordance with publication U.S. Pat. No. 5,096,959. For the purposes of disclosure, the resins set out in the publication U.S. Pat. No. 5,096,959 B1 filed on Oct. 30, 1990 at the Patent Office of the United States of America (USPTO) with the application No. 609,024 are incorporated in the present specification. These alkyd resins are modified with cycloaliphatic polycarboxylic acid, with cyclohexanedicarboxylic and cyclopentanedicarboxylic acids in particular being suitable for the modification.

Additionally it is possible to use alkyd resins which have been modified with polyethylene glycol. A large number of patents describe the preparation of water-emulsifiable alkyd resins by modification with polyethylene glycol (PEG). In the majority of processes about 10% to 30% of PEG is incorporated into the alkyd resin directly by esterification or transesterification (see inter alia U.S. Pat. Nos. 2,634,245; 2,853,459; 3,133,032; 3,223,659; 3,379,548; 3,437,615; 3,437,618; 3,442,835; 3,457,206; 3,639,315; German laid-open specification 14 95 032, or British Patents Nos. 1,038,696 and 1,044,821).

Preferred alkyd resins modified with polyethylene glycol are known from sources including publication EP-A-0 029 145. For the purposes of disclosure, the resins set out in the publication EP-A-0 029 145 filed on Oct. 30, 1980 at the European Patent Office with the application number EP 80106672.1 are incorporated in the present specification. According to that publication it is possible first to react a polyethylene glycol with carboxylic acid containing epoxide groups. The resulting reaction product can then be used in the reaction mixture for preparing the alkyd resin. Preferred polyethylene glycols for modifying the alkyd resins have a number-average molecular weight of 500 to 5000 g/mol, for example.

Particularly preferred polyethylene glycol-modified alkyd resins may additionally be modified with copolymers which are obtainable by polymerizing methacrylic acid, unsaturated fatty acids and vinyl and/or vinylidene compounds.

Additionally judicious are alkyd resins which have been modified with urethane groups. The alkyd resins of this kind are set out in WO 2006/092211 and EP-A-1 533 342, among others.

In one judicious embodiment it is possible to use the urethane alkyd resins that are described in EP-A-1 533 342 and comprise units derived from unsaturated fatty acids A1, aliphatic or aromatic or aromatic-aliphatic monocarboxylic acids A2 that are free from olefinic double bonds, cycloaliphatic dicarboxylic acids A3 or their anhydrides, at least trihydric and preferably at least tetrahydric alcohols A4, and aromatic or aliphatic polyfunctional, especially difunctional, isocyanates A5. The urethane alkyd resin is prepared preferably in a two-stage reaction, with components Al to A4 being esterified in the first stage, the acid number of the product of the first stage being preferably not more than 10 mg/g, more preferably not more than 5 mg/g. In the second stage the hydroxyl-containing product of the first stage is reacted with the isocyanate A5, with addition of a small amount (up to 1% of the mass of the product of the first stage, preferably up to 0.5% of its mass) of a tertiary amine, in a molecular enlargement reaction. Preferred urethane alkyd resins have a Staudinger index, measured in chloroform at 23° C., of at least 9 cm³/g, preferably at least 11 cm³/g.

For the purposes of disclosure, the resins set out in publication EP-A-1 533 342, filed on Nov. 9, 2004 at the European Patent Office with the application number EP 04026511.8, are included in the present application.

With preference it is possible to use urethane alkyd resins which are obtainable by reacting polyhydric alcohols A′, modified fatty acids B′, fatty acids C′ and polyfunctional isocyanates D′. The modified fatty acids B′ can be prepared by reacting unsaturated fatty acids B1′ with unsaturated carboxylic acids B2′. These urethane alkyds are known from publications including WO 2006/092211. For the purposes of disclosure, the resins set out in publication WO 2006/092211, filed on Feb. 20, 2006 at the European Patent Office with the application number PCT/EP2006/001503, are included in the present specification. The modified fatty acid B′ preferably has an acid number of at least 80 mg/g. With particular preference the increase in acid number as a result of the grafting is in the range from 80 mg/g to 250 mg/g, and very preferably in the range from 100 mg/g to 150 mg/g, the acid number being determinable in accordance with DIN EN ISO 2114. The iodine number of the fatty acids C′ used for preparing the urethane alkyd resins is preferably at least 80 g/100 g and preferably at least 120 g/100 g. For preparing the urethane alkyd resin described in WO 2006/092211, in general, first components A′, B′ and C′ are reacted, the condensate preferably having a hydroxy functionality of at least 1.9, more preferably at least 2. Furthermore, the condensate may contain groups derived from polybasic carboxylic acids, especially the above-described dicarboxylic and tricarboxylic acids. This condensate is subsequently reacted with a polyfunctional isocyanate. The preferred polyfunctional isocyanates include, among others, 2,4- and 2,6-toluylene diisocyanate and also the technical mixtures thereof, bis(4-isocyanatophenyl)methane, isophorone diisocyanate, bis(4-isocyanatocyclohexyl)methane and 1,6-diisocyanatohexane, and the biurets, allophanates and isocyanurates derived therefrom.

Besides the above-described conventional alkyd resins, prepared using, generally, polycarboxylic acids, it is also possible to use further alkyd resins, as has already been set out above. These include in particular alkyd resins which are based on urethanes. These urethane alkyd resins can be obtained for example by reaction of polyhydric alcohols with polyfunctional isocyanates. Preferred urethane resins are known for example from EP-A-1 129 147. They can be obtained, for example, by reaction of amide ester diols with polyols and polyfunctional isocyanates. The amide ester diols for use in accordance with EP-A-1 129 147 can be obtained by reacting vegetable oils with N,N-dialkanolamines.

According to one preferred aspect of the present invention, the alkyd resin may have an zo iodine number in accordance with DIN 53241 of at least 1 g iodine/100 g, preferably of at least 10 g iodine/100 g, more preferably at least 15 g iodine/100 g. According to one particular aspect of the present invention, the iodine number of the alkyd resin may lie in the range from 2 to 100 g iodine per 100 g alkyd resin, more preferably 15 to 50 g iodine per 100 g alkyd resin. The iodine number may be determined on the basis of a dispersion, in which case the value is based on the solids content.

Judiciously the alkyd resin may have an acid number in the range from 0.1 to 100 mg KOH/g, preferably 1 to 40 mg KOH/g and very preferably in the range from 2 to 10 mg KOH/g. The acid number can be determined in accordance with DIN EN ISO 2114 from a dispersion, in which case the value is based on the solids content.

The alkyd resin may preferably have a hydroxyl number in the range from 0 to 400 mg KOH/g, more preferably 1 to 200 mg KOH/g and very preferably in the range from 3 to 150 mg KOH/g. The hydroxyl number can be determined in accordance with DIN EN ISO 4629 from a dispersion, in which case the value is based on the solids content.

The preparation of the alkyd resins is well established and is accomplished by condensing the above-recited alcohols and acids, it being possible for modification to take place both during this condensation and after this condensation. Reference in this context is made in particular to the literature set out above.

In the coating materials of the invention it is possible to use the above-recited alkyd resins without modification, but together with polymers of the invention. With regard to the modification it is noted that it can be achieved preferably by polymerizing a (meth)acrylate monomer of formula (I) or a monomer mixture of the invention, with useful information concerning the reaction regime being found in publications including EP-A-0 083 137, the alkyd resins and reaction conditions set out in publication EP-A-0 083 137, filed on Dec. 21, 1987 at the European Patent Office with the application number 82201642.4, being incorporated for purposes of disclosure into the present specification.

The coating material preferably comprises only small amounts of environmentally hazardous solvents, with aqueous dispersions representing particularly preferred coating materials. The aqueous dispersions preferably have a solids content in the range from 10% to 70% by weight, more preferably 20% to 60% by weight. The dynamic viscosity of the dispersion is dependent on the solids content and the particle size and may encompass a wide range. Thus in the case of fine-particle dispersions with a high polymer content the dynamic viscosity may in some cases be more than 10 000 mPas. Judiciously the dynamic viscosity is usually in the range from 10 to 4000 mPas, preferably 10 to 1000 mPas and very preferably 10 to 500 mPas, measured in accordance with DIN EN ISO 2555 at 25° C. (Brookfield).

Additionally the aqueous dispersions of the invention may be provided in a known manner with additives or further components for adapting the properties of the coating material to specific requirements. These adjuvants include, more particularly, drying assistants, known as siccatives, and flow improvers, pigments and dyes.

The coating materials of the invention preferably have a minimum film formation temperature of not more than 50° C., with particular preference not more than 35° C. and very particular preference not more than 25° C., a temperature which can be measured in accordance with DIN ISO 2115.

In accordance with one preferred aspect of the present invention it is possible for a coating material of the invention, more particularly an aqueous dispersion, to have an iodine number in accordance with DIN 53241 of at least 0.1 g iodine/100 g, preferably of at least 10 g iodine/100 g, more preferably of at least 15 g iodine/100 g. The iodine number can be determined on the basis of a dispersion, in which case the value is based on the solids content.

Judiciously the coating material, preferably an aqueous dispersion, may have an acid number in the range 0.1 to 100 mg KOH/g, preferably 1 to 40 mg KOH/g and very preferably in the range from 2 to 10 mg KOH/g. The acid number may be determined in accordance with DIN EN ISO 2114 on the basis of a dispersion, in which case the value is based on the solids content.

The hydroxyl number of a coating material of the invention, more particularly of an aqueous dispersion, may lie preferably in the range from 0 to 400 mg KOH/g, more preferably 1 to 200 mg KOH/g and very preferably in the range from 3 to 150 mg KOH/g. The hydroxyl number may be determined in accordance with DIN EN ISO 4629 on the basis of a dispersion, in which case the value is based on the solids content.

The coating materials of the invention do not require siccatives, although such additives may be included as an optional constituent in the compositions. It may be expedient to use siccatives more particularly in coating materials which can be oxidatively crosslinked. With particular preference it is possible to add siccatives to the aqueous dispersions. These siccatives include, more particularly, organometallic compounds, examples being metal soaps of transition metals, such as cobalt, manganese, lead and zirconium, for example; alkali metals or alkaline earth metals, such as lithium, potassium and calcium, for example. Examples that may be mentioned include cobalt naphthalate and cobalt acetate. The siccatives can be used individually or as a mixture, in which case particular preference is given more particularly to mixtures which comprise cobalt salts, zirconium salts and lithium salts.

The polymers of the present invention can be used more particularly in coating materials or as an adjuvant. Such materials include, more particularly, paints and varnishes, impregnating compositions, adhesives and/or primer systems. With particular preference the coating materials, especially the aqueous dispersions, can be employed for producing paints, varnishes or impregnating compositions for applications on wood and/or metal.

The coatings obtainable from the coating materials of the invention exhibit high solvent resistance; more particularly, only small fractions are dissolved from the coating by solvents. Preferred coatings exhibit a high resistance, more particularly, to methyl isobutyl ketone (MIBK). Hence the weight loss after treatment with MIBK amounts preferably to not more than 50% by weight, more preferably not more than 35% by weight. The absorption of MIBK amounts preferably to not more than 1000% by weight, with particular preference not more than 600% by weight, based on the weight of the coating used. These values are measured at a temperature of approximately 25° C. and over an exposure time of at least 4 hours, the coating subjected to measurement being a fully dried coating which has been crosslinked.

The coatings obtained from the coating materials of the invention display a high mechanical stability. The pendulum hardness is preferably at least 15 s, more preferably at least 25 s, measured in accordance with DIN ISO 1522.

Besides the emulsion polymers, the dispersions of the invention may also comprise further constituents.

The crosslinking of the polymers and/or coating materials of the invention may be accomplished by addition of crosslinking agents. Suitability for this purpose is possessed by nucleophilic compounds which have a multiple functionality. Particularly suitable compounds include diamines, an example being 2,2′-(ethylenedioxy)diethylamine (Jeffamine® XTJ-504, CAS 929-59-9) and dihydrazides, an example being adipic dihydrazide (ADH).

Particularly preferred crosslinking agents are set out in publications including WO 94/25433, filed on 25.04.1994 at the European Patent Office with the application number PCT/EP94/01283, reference being made to this publication for disclosure purposes, and the crosslinking agents disclosed therein being incorporated into the present specification.

The fraction of crosslinking agent is not critical per se, the amount of crosslinking agent being ascertained preferably on the basis of the polymer. With particular preference the molar ratio of crosslinkable groups present in the polymer to the reactive groups of the crosslinking agent is in the range from 10:1 to 1:10, more preferably 4:1 to 1:4 and very preferably in the range from 2:1 to 1:2.

The present invention will be illustrated below with reference to an inventive example and comparative examples, without any intention thereby to restrict the invention.

### EXAMPLE 1

### PREPARATION EXAMPLE

10 mmol of allyl methacrylate in 25 ml of THF were fed from a buret maintained at a constant temperature to a 100 ml Parr autoclave with pressure maintenance device and were reacted at a pressure of 10 bar with a CO/H₂ gas mixture which had a molar ratio of 1:1, in the presence of 0.1 mol % of Rh(acac)(CO)₂ and 0.2 mol % of Xantphos, based in each case on allyl methacrylate, at a temperature of 65° C. for a reaction time of 20 hours. A yield of 90% was obtained, the reaction mixture comprising 4-oxobutyl methacrylate and 2-methyl-3-oxopropyl methacrylate. The ratio of 4-oxobutyl methacrylate to 2-methyl-3-oxopropyl methacrylate was 92:8.

### EXAMPLE 2

### PREPARATION EAMPLE

Example 1 was essentially repeated, but using 0.02 mol % of Rh(acac)(CO)₂ and 0.04 mol % of Xantphos, based in each case on allyl methacrylate. The yield was 85% and the ratio of 4-oxobutyl methacrylate to 2-methyl-3-oxopropyl methacrylate was 92:8.

### EXAMPLE 3

### PREPARATION EXAMPLE

Example 1 was essentially repeated, but operating at a pressure of 20 bar. The yield was 95% and the ratio of 4-oxobutyl methacrylate to 2-methyl-3-oxopropyl methacrylate was 88:12.

### EXAMPLE 4

### PREPARATION EXAMPLE

50 mmol of allyl methacrylate in 25 ml of THF were fed from a buret maintained at a constant temperature to a 100 ml Parr autoclave with pressure maintenance device and were reacted at a pressure of 20 bar with a CO/H₂ gas mixture which had a molar ratio of 1:1, in the presence of 0.02 mol % (2.5 mg) of Rh(acac)(CO)₂ and 0.04 mol % of Biphephos, based in each case on allyl methacrylate, at a temperature of 65° C. for a reaction time of 2 hours. A yield of 99% was obtained, the reaction mixture comprising 4-oxobutyl methacrylate and 2-methyl-3-oxopropyl methacrylate. The ratio of 4-oxobutyl methacrylate to 2-methyl-3-oxopropyl methacrylate was 60.40.

### EXAMPLE 5

### PREPARATION EXAMPLE

Example 4 was essentially repeated, but operating at a pressure of 10 bar. The yield was 99% and the ratio of 4-oxobutyl methacrylate to 2-methyl-3-oxopropyl methacrylate was 68:32.

### EXAMPLE 6

### PREPARATION EXAMPLE

Example 5 was essentially repeated, but using 0.02 mol % of Rh(acac)(CO)₂ and 0.12 mol % of Biphephos, based in each case on allyl methacrylate. The yield was 99% and the ratio of 4-oxobutyl methacrylate to 2-methyl-3-oxopropyl methacrylate was 73:27.

### EXAMPLE 7

### PREPARATION OF A DISPERSION

BuA-co-MMA-ObMA-MAA=53.9-43.1-2-1

First of all, in a 1 l PE beaker, 107.8 g of butyl acrylate (BuA), 86.2 g of methyl methacrylate (MMA), 4 g of oxobutyl methacrylate (ObMA), 2 g of methacrylic acid (MAA), 0.6 g of ammonium peroxodisulphate (APS), 6.0 g of Disponil FES 32 (30% form) and 179.6 g of water were emulsified using an Ultra-Turrax at 4000 rpm for 3 minutes.

A 1 l glass reactor which could be maintained at a certain temperature using a water bath and was equipped with a paddle stirrer was charged with 110 g of water and 0.15 g of Disponil FES 32 (30% form) and this initial charge was heated to 80° C. and admixed with 0.15 g of ammonium peroxodisulphate (APS) in solution in 10 g of water. 5 minutes after the addition of the APS, the above-prepared emulsion was metered in over the course of 240 minutes (interval: 3 minutes' feed, 4 minutes' wait, 237 minutes' feed of remainder).

After the end of the feed, stirring was continued at 80° C. for 1 hour. Thereafter the dispersion was cooled to room temperature and filtered off through a VA sieve with a mesh size of 0.09 mm.

The emulsion prepared had a solids content of 40±1%, a pH of 2.6, a viscosity of 16 mPas and a r_{N5} value of 85 nm.

### EXAMPLE 8

### PREPARATION OF A DISPERSION

BuA-co-MMA-ObMA-MAA=52.8-42.2-4-1

First of all, in a 1 l PE beaker, 105.6 g of butyl acrylate (BuA), 84.4 g of methyl methacrylate (MMA), 8 g of oxobutyl methacrylate (ObMA), 2 g of methacrylic acid (MAA), 0.6 g of ammonium peroxodisulphate (APS), 6.0 g of Disponil FES 32 (30% form) and 179.6 g of water were emulsified using an Ultra-Turrax at 4000 rpm for 3 minutes.

A 1 l glass reactor which could be maintained at a certain temperature using a water bath and was equipped with a paddle stirrer was charged with 110 g of water and 0.15 g of Disponil FES 32 (30% form) and this initial charge was heated to 80° C. and admixed with 0.15 g of ammonium peroxodisulphate (APS) in solution in 10 g of water. 5 minutes after the addition of the APS, the above-prepared emulsion was metered in over the course of 240 minutes (interval: 3 minutes' feed, 4 minutes' wait, 237 minutes' feed of remainder).

After the end of the feed, stirring was continued at 80° C. for 1 hour. Thereafter the dispersion was cooled to room temperature and filtered off through a VA sieve with a mesh size of 0.09 mm.

The emulsion prepared had a solids content of 40±1%, a pH of 2.5, a viscosity of 14 mPas and a r_{N5} value of 84 nm.

### EXAMPLE 9

### PREPARATION OF A DISPERSION

BuA-co-MMA-ObMA-MAA=51.7-41.3-6-1

First of all, in a 1 l PE beaker, 103.4 g of butyl acrylate (BuA), 82.6 g of methyl methacrylate (MMA), 12 g of oxobutyl methacrylate (ObMA), 2 g of methacrylic acid (MAA), 0.6 g of ammonium peroxodisulphate (APS), 6.0 g of Disponil FES 32 (30% form) and 179.6 g of water were emulsified using an Ultra-Turrax at 4000 rpm for 3 minutes.

A 1 l glass reactor which could be maintained at a certain temperature using a water bath and was equipped with a paddle stirrer was charged with 110 g of water and 0.15 g of Disponil FES 32 (30% form) and this initial charge was heated to 80° C. and admixed with 0.15 g of ammonium peroxodisulphate (APS) in solution in 10 g of water. 5 minutes after the addition of the APS, the above-prepared emulsion was metered in over the course of 240 minutes (interval: 3 minutes' feed, 4 minutes' wait, 237 minutes' feed of remainder).

After the end of the feed, stirring was continued at 80° C. for 1 hour. Thereafter the dispersion was cooled to room temperature and filtered off through a VA sieve with a mesh size of 0.09 mm.

The emulsion prepared had a solids content of 40±1%, a pH of 2.5, a viscosity of 14 mPas and a r_{N5} value of 90 nm.

### EXAMPLES 10 to 12

### DIHYDRAZIDE CROSSLINKING OF DISPERSIONS OBTAINED IN EXAMPLES 7 TO 9

The dispersions obtained in examples 7 to 9 were crosslinked equimolarly with adipic dihydrazide (ADH). This was done by adding a 15% strength ADH solution dropwise to the dispersion, with stirring, continuing stirring for 2 hours, and drying a film at room temperature.

The properties of the resultant coating material were investigated by means of different methods. Tests relating to the solvent resistance, water absorption and hardness were carried out on dried films for this purpose.

The solvent resistance was determined using methyl isobutyl ketone (MIBK), a sample (A) being swollen with MIBK for 4 hours at room temperature. The sample was then removed from the solvent, excess solvent was removed, and the weight was measured. Subsequently the sample was dried at about 140° C. for 1 hour (B). The difference in weight between A and B is used to calculate the fraction of the sample that has been removed by the solvent. The swelling was calculated on the basis of the weight of sample B, freed from soluble fractions, and is referred to in the text below as “true swelling”.

To determine the water absorption, a sample was swollen in water at room temperature for 24 hours. The procedure was then the same as that for determining the solvent resistance. The result is referred to in the text below as “true water absorption”.

Additionally, a furniture test was carried out in the same way as in DIN 68861-1. On the evaluation scale of 1-5, 5 denotes no visible change and 1 denotes severe change or destruction of the area under test.

The results obtained are set out in Table 1. Example 10 was obtained using a dispersion of example 7, example 11 using a dispersion of example 8, and example 12 using a dispersion of example 9.

### EXAMPLES 13 to 15

### DIAMINE CROSSLINKING OF DISPERSIONS OBTAINED IN EXAMPLES 7 to 9

The dispersions obtained in examples 7 to 9 were crosslinked equimolarly with 2,2′-(ethylenedioxy)diethylamine (Jeffamine® XTJ-504, CAS 929-59-9). This was done by adding the diamine dropwise to the dispersion, with stirring, continuing stirring for 2 hours, and drying a film at room temperature.

The properties of the resultant coating material were investigated by means of different methods. Tests relating to the solvent resistance, water absorption and hardness were carried out on dried films for this purpose.

The solvent resistance was determined using methyl isobutyl ketone (MIBK), a sample being swollen with MIBK for 4 hours at room temperature. The sample was then removed from the solvent, and excess solvent was removed. Subsequently the sample was dried at about 140° C. for 1 hour. The loss in weight is used to calculate the fraction of the sample removed by the solvent.

To determine the water absorption, a sample was swollen in water at room temperature for 24 hours. Subsequently the sample was removed from the water, and excess water was removed. Then the sample was dried at about 140° C. for 1 hour. The fraction of the sample removed by the water is calculated from the weight loss.

The results obtained are set out in Table 1. Example 13 was obtained using a dispersion of example 7, example 14 using a dispersion of example 8, and example 15 using a dispersion of example 9.

### COMPARATIVE EXAMPLE 1

BuA-co-MMA-AAEMA-MAA=53.9-43.1-2-1

For comparison, instead of oxobutyl methacrylate, the commercially available acetoacetoxyethyl methacrylate (AAEMA) was incorporated into dispersions and crosslinked with ADH.

First of all, in a 2 l PE beaker, 215.6 g of butyl acrylate (BuA), 172.4 g of methyl methacrylate (MMA), 8 g of acetoacetoxyethyl methacrylate (AAEMA), 4 g of methacrylic acid (MAA), 1.2 g of ammonium peroxodisulphate (APS), 12.0 g of Disponil FES 32 (30% form) and 359.18 g of water were emulsified using an Ultra-Turrax at 4000 rpm for 3 minutes.

A 2 l glass reactor which could be maintained at a certain temperature using a water bath and was equipped with a paddle stirrer was charged with 230 g of water and 0.3 g of Disponil FES 32 (30% form) and this initial charge was heated to 80° C. and admixed with 0.3 g of ammonium peroxodisulphate (APS) in solution in 10 g of water. 5 minutes after the addition of the APS, the above-prepared emulsion was metered in over the course of 240 minutes (interval: 3 minutes' feed, 4 minutes' wait, 237 minutes' feed of remainder).

After the end of the feed, stirring was continued at 80° C. for 1 hour. Thereafter the dispersion was cooled to room temperature and filtered off through a VA sieve with a mesh size of 0.09 mm.

Experiments relating to the solvent resistance, water absorption and scratch resistance were carried out on dried films.

The emulsion prepared had a solids content of 40±1%, a pH of 2.4 and a r_{N5} value of 87 nm.

### COMPARATIVE EXAMPLE 2

BuA-co-MMA-AAEMA-MAA=52.8-42.2-4-1

For comparison, instead of oxobutyl methacrylate, the commercially available acetoacetoxyethyl methacrylate (AAEMA) was incorporated into dispersions and crosslinked with ADH.

First of all, in a 2 l PE beaker, 211.1 g of butyl acrylate (BuA), 168.8 g of methyl methacrylate (MMA), 16 g of acetoacetoxyethyl methacrylate (AAEMA), 4 g of methacrylic acid (MAA), 1.2 g of ammonium peroxodisulphate (APS), 12.0 g of Disponil FES 32 (30% form) and 359.18 g of water were emulsified using an Ultra-Turrax at 4000 rpm for 3 minutes.

A 2 l glass reactor which could be maintained at a certain temperature using a water bath and was equipped with a paddle stirrer was charged with 230 g of water and 0.3 g of Disponil FES 32 (30% form) and this initial charge was heated to 80° C. and admixed with 0.3 g of ammonium peroxodisulphate (APS) in solution in 10 g of water. 5 minutes after the addition of the APS, the above-prepared emulsion was metered in over the course of 240 minutes (interval: 3 minutes' feed, 4 minutes' wait, 237 minutes' feed of remainder).

After the end of the feed, stirring was continued at 80° C. for 1 hour. Thereafter the dispersion was cooled to room temperature and filtered off through a VA sieve with a mesh size of 0.09 mm.

Experiments relating to the solvent resistance, water absorption and scratch resistance were carried out on dried films.

The emulsion prepared had a solids content of 40±1%, a pH of 2.4 and a r_{N5} value of 82 nm.

### COMPARATIVE EXAMPLE 3

BuA-co-MMA-AAEMA-MAA=51.7-41.3-6-1

For comparison, instead of oxobutyl methacrylate, the commercially available acetoacetoxyethyl methacrylate (AAEMA) was incorporated into dispersions and crosslinked with ADH.

First of all, in a 2 l PE beaker, 206.8 g of butyl acrylate (BuA), 165.2 g of methyl methacrylate (MMA), 24 g of acetoacetoxyethyl methacrylate (AAEMA), 4 g of methacrylic acid (MAA), 1.2 g of ammonium peroxodisulphate (APS), 12.0 g of Disponil FES 32 (30% form) and 359.18 g of water were emulsified using an Ultra-Turrax at 4000 rpm for 3 minutes.

A 2 l glass reactor which could be maintained at a certain temperature using a water bath and was equipped with a paddle stirrer was charged with 230 g of water and 0.3 g of Disponil FES 32 (30% form) and this initial charge was heated to 80° C. and admixed with 0.3 g of ammonium peroxodisulphate (APS) in solution in 10 g of water. 5 minutes after the addition of the APS, the above-prepared emulsion was metered in over the course of 240 minutes (interval: 3 minutes' feed, 4 minutes' wait, 237 minutes' feed of remainder).

After the end of the feed, stirring was continued at 80° C. for 1 hour. Thereafter the dispersion was cooled to room temperature and filtered off through a VA sieve with a mesh size of 0.09 mm. Experiments relating to the solvent resistance, water absorption and scratch resistance were carried out on dried films.

The emulsion prepared had a solids content of 40±1%, a pH of 2.4 and a r_{N5} value of 86 nm.

Comparative examples 4 to 6 (Dihydrazide crosslinking of dispersions obtained in comparative examples 1 to 3)

The dispersions obtained in comparative examples 1 to 3 were crosslinked equimolarly with adipic dihydrazide (ADH). This was done by adding a 15% strength ADH solution dropwise to the dispersion, with stirring, continuing stirring for 30 minutes, and drying a film at room temperature.

The properties of the resultant coating material were investigated by means of different methods. Tests relating to the solvent resistance, water absorption and hardness were carried out on dried films for this purpose.

The solvent resistance was determined using methyl isobutyl ketone (MIBK), a sample being swollen with MIBK for 4 hours at room temperature. The sample was then removed from the solvent, and excess solvent was removed. Subsequently the sample was dried at about 140° C. for 1 hour. The loss in weight is used to calculate the fraction of the sample removed by the solvent.

To determine the water absorption, a sample was swollen in water at room temperature for 24 hours. Subsequently the sample was removed from the water, and excess water was removed. Then the sample was dried at about 140° C. for 1 hour. The fraction of the sample removed by the water is calculated from the weight loss.

The results obtained are set out in Table 1. Comparative example 4 was obtained using a dispersion of comparative example 1, comparative example 5 using a dispersion of comparative example 2, and comparative example 6 using a dispersion of comparative example 3.

TABLE 1
Results of the investigations of properties

| | Example 10 | Example 11 | Example 12 |
|---|---|---|---|
| Pendulum hardness [s] | 22.4 | 28.7 | 45.5 |
| True swelling in MIBK [%] | 914 | 540 | 431 |
| Weight loss in MIBK [%] | 17.4 | 11.8 | 8.3 |
| True swelling in ethanol [%] | 156 | 129 | 116 |
| Weight loss in ethanol [%] | 7.3 | 5.3 | 3.5 |
| True water absorption of the | 10.1 | 12.3 | 12.5 |
| film after 24 h [%] | | | |
| Tensile strength [MPa] | 10.9 | 15.8 | 20.4 |
| Elongation at break [%] | 304.9 | 207.7 | 166.5 |
| Furniture test, ethanol, after | 5/5 | 5/5 | 5/5 |
| 1 h/16 h | | | |
| Furniture test, HOAc, after | 5/5 | 5/5 | 5/5 |
| 1 h/16 h | | | |

TABLE 1
Results of the investigations of properties

| | Comparative | Comparative | Comparative |
|---|---|---|---|
| | example 4 | example 5 | example 6 |
| Pendulum hardness [s] | 9.8 | 13.3 | 21.7 |
| True swelling in MIBK [%] | 1311 | 835 | 814 |
| Weight loss in MIBK [%] | 15.9 | 9.6 | 13.6 |
| True swelling in ethanol [%] | 141 | 114 | 138 |
| Weight loss in ethanol [%] | 5.3 | 3.3 | 14.2 |
| True water absorption of the | 12.0 | 9.0 | 10.9 |
| film after 24 h [%] | | | |
| Tensile strength [MPa] | 7.7 | 10.2 | 12.2 |
| Elongation at break [%] | 429.2 | 280.7 | 233.4 |
| Furniture test, ethanol, after | 5/5 | 5/5 | 1/1 |
| 1 h/16 h | | | |
| Furniture test, HOAc, after | 5/4 | 5/3 | 5/5 |
| 1 h/16 h | | | |
| | Example 13 | Example 14 | Example 15 |
| Pendulum hardness [s] | 19.6 | 25.9 | 38.5 |
| True swelling in MIBK [%] | 309 | 217 | 195 |
| Weight loss in MIBK [%] | 6.2 | 3.0 | 2.4 |
| True swelling in ethanol [%] | 101 | 98 | 124 |
| Weight loss in ethanol [%] | 5.6 | 4.7 | 4.2 |
| True water absorption of the | 13.2 | 11.4 | 12.2 |
| film after 24 h [%] | | | |
| Tensile strength [MPa] | 5.3 | 6.7 | 9.5 |
| Elongation at break [%] | 236.9 | 147.2 | 111.0 |
| Furniture test, ethanol, after | 5/3 | 5/5 | 5/5 |
| 1 h/16 h | | | |
| Furniture test, HOAc, after | 5/5 | 5/5 | 5/5 |
| 1 h/16 h | | | |

1. A (meth)acrylate monomer represented by formula (I) 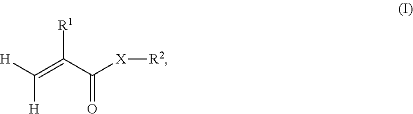
in which R¹ is hydrogen or a methyl group, X is oxygen or a group of the formula NR′ in which R′ is hydrogen or a radical having 1 to 6 carbon atoms, and R² is a radical having 3 to 31 carbon atoms and at least one aldehyde group.

2. The (meth)acrylate monomer according to claim 1, wherein the radical R² has one or two aldehyde groups.

3. The (meth)acrylate monomer according to claim 1, wherein radical R² is alkyl group having 4 to 6 carbon atoms and one aldehyde group.

4. The (meth)acrylate monomer according to claim 3, wherein the monomer is 4-oxobutyl(meth)acrylate or 2-methyl-3-oxopropyl(meth)acrylate.

5. The (meth)acrylate monomer according to claim 1, wherein the (meth)acrylate monomer is of the formula (II) 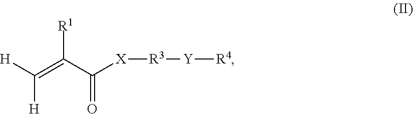
in which R¹ is hydrogen or a methyl group, X is oxygen or a group of the formula NR′ in which R′ is hydrogen or a radical having 1 to 6 carbon atoms, R³ is an alkylene group having 1 to 22 carbon atoms, Y is oxygen, sulphur or a group of the formula NR″ in which R″ is hydrogen or a radical having 1 to 6 carbon atoms, and R⁴ is a radical having 8 carbon atoms and at least one aldehyde group.

6. The (meth)acrylate monomer according to claim 1, wherein the (meth)acrylate monomer has 10 to 25 carbon atoms in the radical R².

7. The (meth)acrylate monomer according to claim 1, wherein the radical R² comprises at least one double bond.

8. A monomer mixture comprising at least one (meth)acrylate monomer according to claim 1.

9. The monomer mixture according to claim 8, wherein the monomer mixture comprises monomers A which comprise ester groups that are different from the (meth)acrylate.

10. The monomer mixture according to claim 9, wherein the monomer mixture comprises a (meth)acrylate, a fumarate, a maleate and/or vinyl acetate.

11. The monomer mixture according to claim 9, wherein the monomer mixture comprises a (meth)acrylate of formula (III) 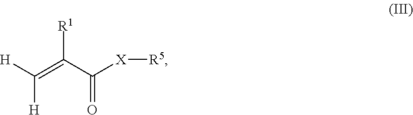
in which X is oxygen or a group of the formula NW in which R′ is hydrogen or a radical having 1 to 6 carbon atoms, and R⁵ is an unsaturated radical having at least one double bond and 2 to 30 carbon atoms.

12. The monomer mixture according to claim 8, wherein the monomer mixture comprises and at least one styrene monomer.

13. The monomer mixture according to claim 8, wherein the monomer mixture comprises at least one monomer having an acid group.

14. The monomer mixture according to claim 8, wherein the monomer mixture comprises
0.1% to 90% by weight of (meth)acrylate monomer of formula (I),
10% to 99.9% by weight of monomer A comprising ester groups,
0% to 10% by weight of monomer having an acid group,
0% to 70% by weight of styrene monomers and
0% to 50% by weight of further comonomers.

15. A polymer comprising at least one unit derived from a (meth)acrylate monomer according to claim 1.

16. The polymer according to claim 15, wherein the polymer is obtained obtainable by a process comprising polymerizing a monomer mixture comprising the (meth)acrylate monomer.

17. The polymer according to claim 15, wherein the polymer has a glass transition temperature in the range from −60 to 120° C.

18. The polymer according to claim 15, wherein the polymer is an emulsion polymer.

19. A coating material comprising a polymer according to claim 15.

20. A coating material comprising an alkyd resin which has been modified with the (meth)acrylate monomer.

21. The coating material according to claim 19, wherein the coating material comprises an alkyd resin and a monomer mixture comprising the (meth)acrylate monomer and a monomer A which is different from the (meth)acrylate monomer and comprises an ester group.

22. The coating material according to claim 19, wherein the coating material is an aqueous dispersion.

23. A process for preparing a monomer according to claim 1, comprising reacting a reactant of the formula (III) 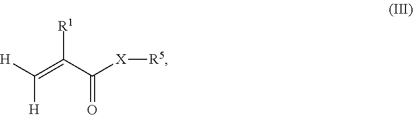
in which X is oxygen or a group of the formula NR′ in which R′ is hydrogen or a radical having 1 to 6 carbon atoms, and R⁵ is an unsaturated radical having at least one double bond and 2 to 24 carbon atoms with carbon monoxide and hydrogen in the presence of a catalyst.

24. The process Process according to claim 23, wherein the catalyst comprises rhodium, iridium, palladium and/or cobalt.

25. The process according to claim 23, wherein the catalyst is a complex which comprises a phosphorus compound as ligand.

26. The process according to claim 23, wherein the reaction is carried out at a temperature in the range from 20 to 250° C., preferably from 40 to 200° C.

27. The process according to claim 23, wherein the reaction is carried out at an overall gas pressure in the range from 1 to 200 bar.

28. The process according to claim 23, wherein the hydrogen pressure at which reaction is carried out is greater than the pressure of the carbon monoxide.

29. The process according to claim 25, wherein the phosphorus compound employed as ligand is used in excess over the metal.

30. The process according to claim 29, wherein the catalyst comprises rhodium, iridium, palladium and/or cobalt, and the ratio of metal to ligand is in the range from 1:1 to 1:1000.

31. The process Process according to claim 23, wherein the reaction is carried out in an inert organic solvent.

32. The process according to claim 23, wherein the reaction is carried out substantially without the use of an inert organic solvent.

33. The process according to claim 23, wherein the reaction is carried out in the presence of a stabilizer.

34. A process for crosslinking a polymer, comprising reacting a polymer according to claim 15 with a diamine and/or a dihydrazide.
